(19) United States
(12) Patent Application Publication
(10) Pub. No.: US 2008/0090242 A1
(43) Pub. Date: Apr. 17, 2008

(54) PANEL OF BIOMARKERS FOR PREDICTION OF FTI EFFICACY

(75) Inventors: Diane Levitan, Tenafly, NJ (US); Andrea Dawn Basso, New City, NY (US); Marvin Bayne, Westfield, NJ (US); Walter Robert Bishop, Pompton Plains, NJ (US); Paul Kirschmeier, Basking Ridge, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/864,163

(22) Filed: Sep. 28, 2007

Related U.S. Application Data
(60) Provisional application No. 60/861,370, filed on Nov. 28, 2006.
(60) Provisional application No. 60/848,147, filed on Sep. 29, 2006.

(51) Int. Cl. C12Q 1/68

(52) U.S. Cl. 435/6

(57) ABSTRACT

The present invention provides, inter alia, methods for selecting a patient with cancer for treatment with a farnesyl protein transferase inhibitor as well as methods for treating said patient.

This application claims the benefit of U.S. provisional patent application No. 60/861,370, filed Nov. 28, 2006; and U.S. provisional patent application No. 60/848,147, filed Sep. 29, 2006; each of which is herein incorporated by reference in its entirety.

### FIELD OF THE INVENTION

The field of the invention concerns, inter alia, methods for selecting patients for treatment with an FPT inhibitor.

### BACKGROUND OF THE INVENTION

Farnesyl protein transferase (FPT) inhibitors (FTIs) are a current area of interest in the treatment and prevention of cancerous conditions. Indeed, there are several FTIs currently in clinical development or on the market. Examples of such FTIs include lonafarnib (Sarasar™; Schering Corporation; Kenilworth, N.J.) and tipifarnib (Zarnestra®; Johnson & Johnson).

Early and effective treatment of cancer is a critical factor affecting the survival of cancer patients. The selection of treatment regimens against which a cancer is resistant delays the onset of effective treatment of the cancer and can leads to growth and spread of the cancer. This, in turn, can have a negative effect on the patient's treatment outcome. Accordingly, the early selection of patients with tumors which are likely to be responsive to a given FTI is of interest. Tumor-specific characteristics that are associated with responsiveness to an FTI, such as the expression of one or more specific genes, can be used as biomarkers for the likelihood of sensitivity to that FTI. Accordingly, patients suffering from tumors expressing any of such biomarkers can be selected for treatment with an FTI. This approach of patient selection has been employed successfully in connection with other cancer treatments. For example, Bunn et al., report selection criteria for patients with non-small cell lung cancer for treatment with an epidermal growth factor receptor (EGFR) tyrosine kinase inhibitor (Clin, Cancer Res. 12: 3652-3656 (2006)). Han at al. identified markers (EGFR mutation, K-ras Mutation and Akt Phosphorylation) pointing to a likelihood of sensitivity to gefitinib (Clin. Cancer Res. 12: 2538-2544 (2006)).

Currently, there is a need in the art for the identification of biomarkers indicating a likelihood of FTI sensitivity.

### SUMMARY OF THE INVENTION

The present invention addresses this need, for example, by provision of the methods of the present invention as set forth herein.

The present invention provides a method for treating a tumor in a patient comprising (a) determining if the tumor is likely to be sensitive to a farnesyl protein transferase inhibitor, wherein the tumor is likely to be sensitive to the inhibitor if at least one biomarker selected from the group consisting of PRL2, claudin-1 (CLDN1), mucin-1 (MUC1), LTB4DH and endothelin-1 (EDN1; ET-1) is underexpressed by a cell in the tumor and/or PDGFRL is overexpressed by a cell in the tumor, relative to expression of the biomarker by a farnesyl protein transferase inhibitor resistant cell; and (b) administering, to said patient, a therapeutically effective amount of a farnesyl protein transferase inhibitor if the tumor is likely to be sensitive. In an embodiment of the invention, the patient is human. In an embodiment of the invention, the patient, has a tumor comprising a cell wherein PRL2 expression is less than that of a farnesyl protein transferase inhibitor resistant cell, is selected. In an embodiment of the invention, PRL2 comprises the nucleotide sequence set forth in SEQ ID NO: 2. In an embodiment of the invention, the farnesyl protein transferase inhibitor resistant cell is T47D or SKOV3. In an embodiment of the invention, the tumor is a member selected from the group consisting of lung cancer, lung adenocarcinoma, non small cell lung cancer, pancreatic cancer, exocrine pancreatic carcinoma, colon cancer, colorectal carcinoma, colon adenocarcinoma, colon adenoma, myeloid leukemia, acute myelogenous leukemia (AML), chronic myelogenous leukemia (CML), and chronic myelomonocytic leukemias (CMML), thyroid follicular cancer, myelodysplastic syndrome (MDS), bladder carcinoma, epidermal carcinoma, melanoma, breast cancer, prostate cancer, head and neck cancer, squamous cell cancer of the head and neck, ovarian cancer, brain cancer, glioma, cancers of mesenchymal origin, fibrosarcomas, rhabdomyosarcomas, sarcomas, tetracarcinomas, neuroblastomas, kidney carcinomas, hepatomas, non-Hodgkin's lymphoma, multiple myeloma, and anaplastic thyroid carcinomas in an embodiment of the invention, the farnesyl protein transferase inhibitor is one or more members selected from the group consisting of: 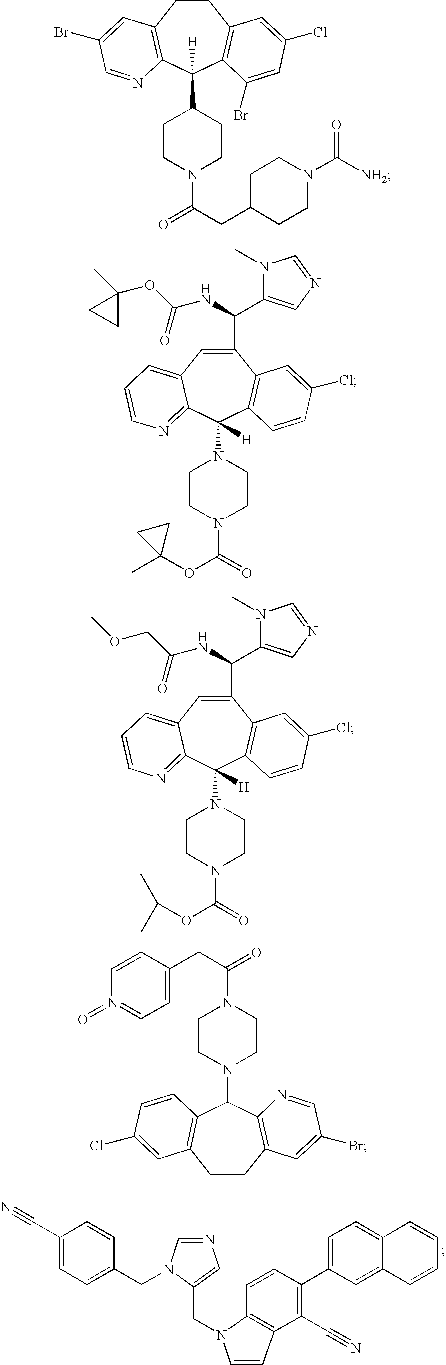 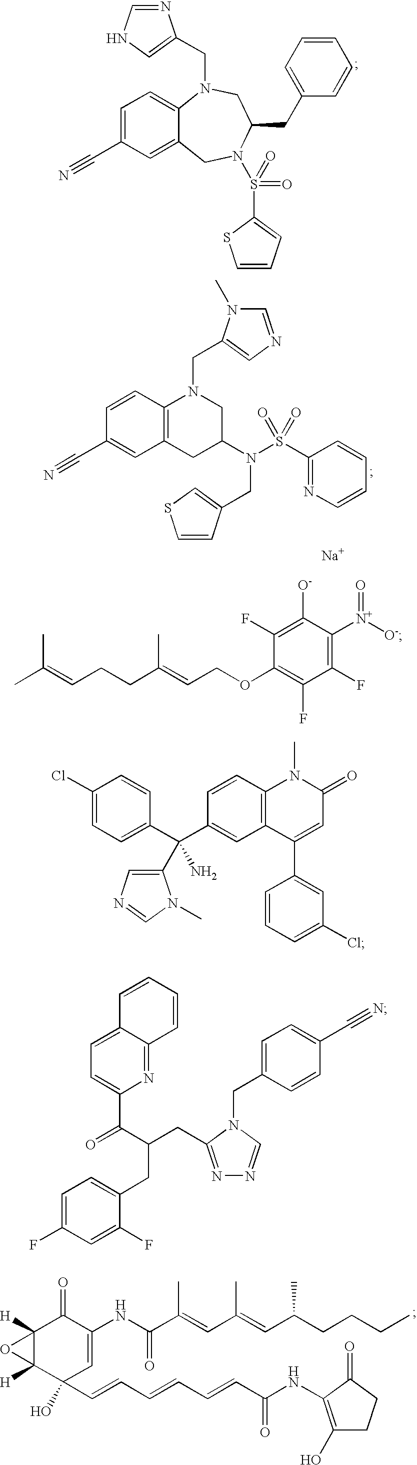 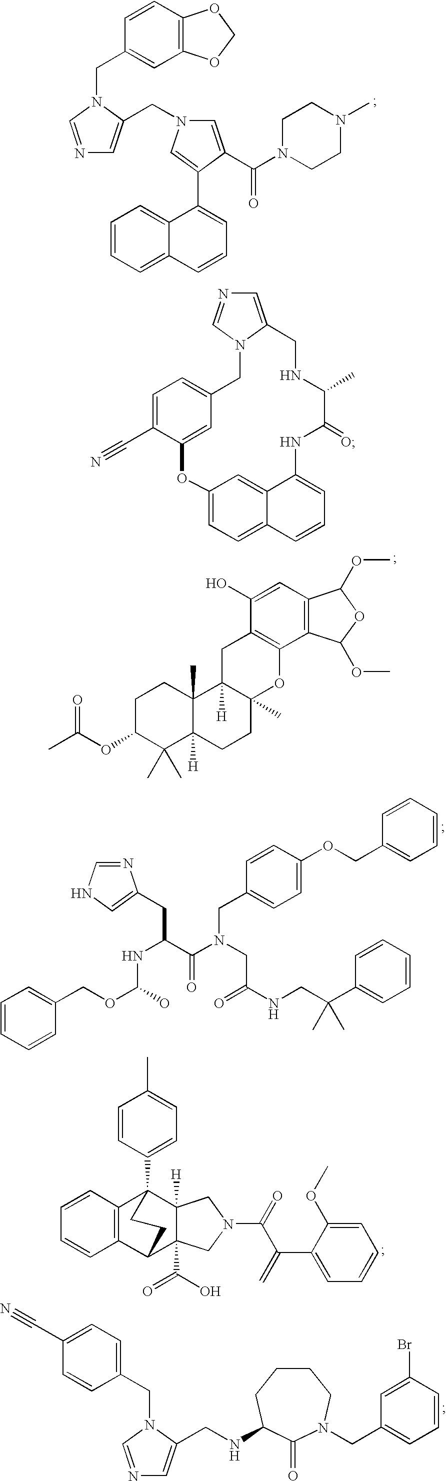 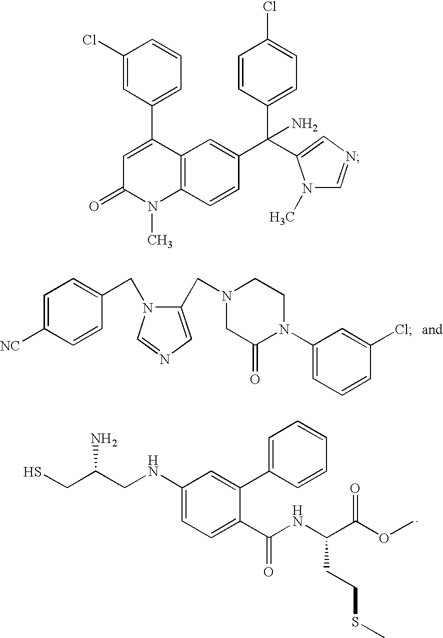
In an embodiment of the invention, the patient is administered the farnesyl protein transferase inhibitor in association with a further chemotherapeutic agent or a further therapeutic procedure. In an embodiment of the invention, the further therapeutic procedure is a member selected from the group consisting of anti-cancer radiation therapy and surgical tumorectomy. In an embodiment of the invention, the further chemotherapeutic agent is one or more members selected from the group consisting of paclitaxel, gemcitabine, trastuzumab, cisplatin, docetaxel, doxorubicin, melphalan and 5-fluorouracil.

The present invention provides a method for assessing whether a farnesyl protein transferase inhibitor inhibits in vitro or in vivo growth or survival of a tumor cell comprising determining if said cell underexpresses PRL2, claudin-1, mucin-1, LT84DH or endothelin-1 and/or overexpresses PDGFRL, relative to farnesyl protein transferase inhibitor resistant cell expression of the biomarker, wherein the inhibitor is determined to inhibit said growth or survival if said underexpression or overexpression is observed. In an embodiment of the invention, expression of the biomarker is assessed by northern blot analysis, real-time polymerase chain reaction (RT-PCR) analysis, western blot analysis, enzyme linked immunosorbent assay (ELISA) analysis, radioimmunoassay analysis (RIA), immunohistochemistry or immunofluorescence. In an embodiment of the invention, the patient is human. In an embodiment of the invention the patient has a tumor comprising a cell wherein PRL2 expression is less than that of a farnesyl protein transferase inhibitor resistant cell, is selected. In an embodiment of the invention, PRL2 comprises the nucleotide sequence set forth in SEQ ID NO, 2. In an embodiment of the invention, the resistant cell is T47D or SKOV3.

The present invention provides a method for selecting a patient with a tumor responsive to a farnesyl protein transferase inhibitor comprising determining if a cell from said tumor underexpresses of PRL2, claudin-1 mucin-1, LTB4DH or endothelin-1 and/or overexpresses PDGFRL, relative to resistant cell expression of the biomarker; wherein the patient is selected if said underexpression or overexpression is observed. In an embodiment of the invention, the resistant cell is T47D or SKOV3. In an embodiment of the invention, the patient is human. In an embodiment of the invention, the patient has a tumor comprising a cell wherein PRL2 expression is less than that of expression of PRL2 in a resistant cell is selected. In an embodiment of the invention, PRL2 comprises the nucleotide sequence set forth in SEQ ID NO: 2. In an embodiment of the invention, the resistant cell is T47D or SKOV3. In an embodiment of the invention, the patient is treated with a farnesyl protein transferase inhibitor and, optionally, a further chemotherapeutic agent. In an embodiment of the invention, the farnesyl protein transferase inhibitor is one or more members selected from the group consisting of: 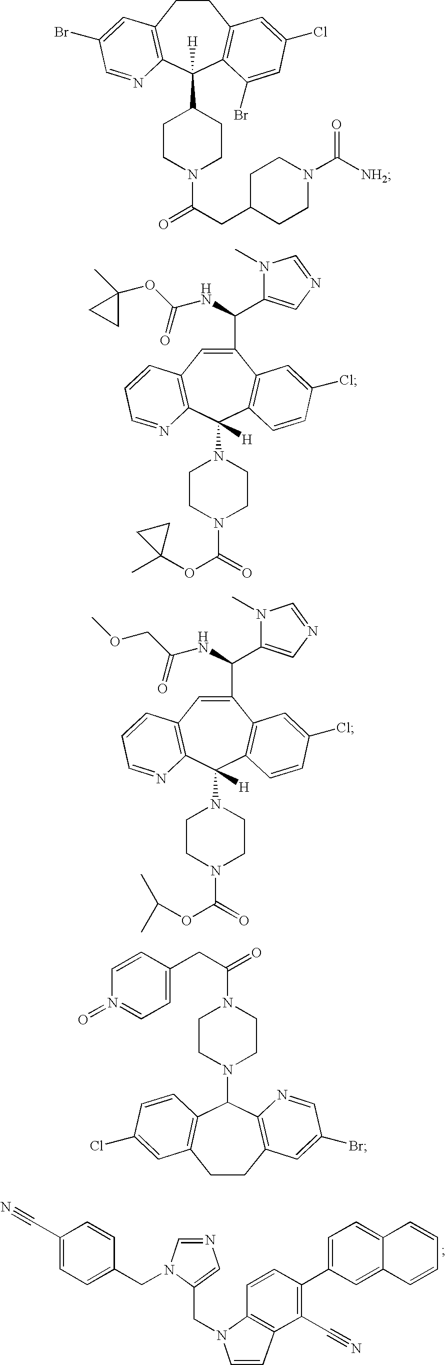 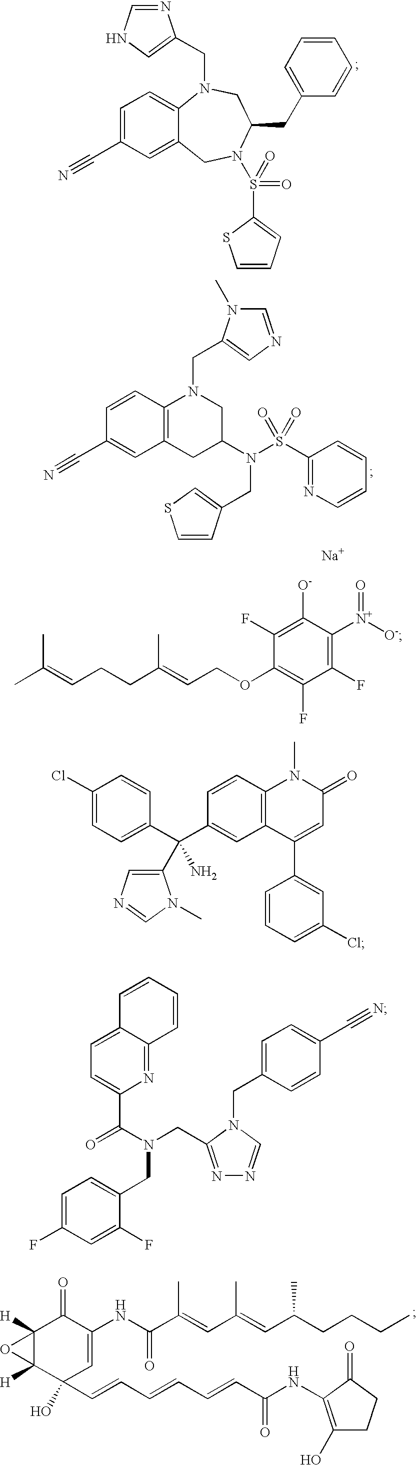 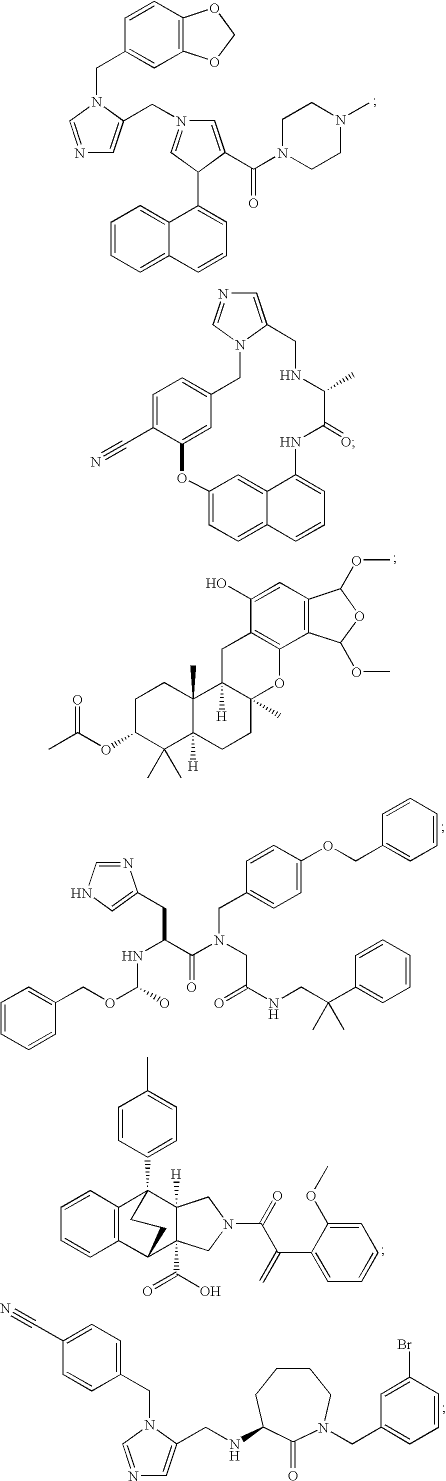 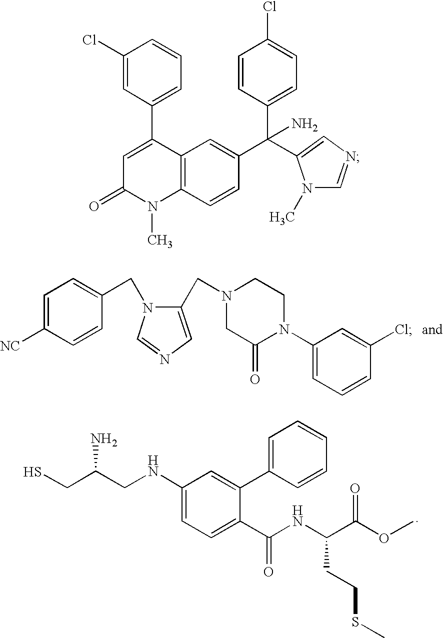
In an embodiment of the invention, the patient is administered the farnesyl protein transferase inhibitor in association with a further therapeutic procedure. In an embodiment of the invention, the further therapeutic procedure is a member selected from the group consisting of anti-cancer radiation therapy and surgical tumorectomy. In an embodiment of the invention, the further chemotherapeutic agent is one or more members selected from the group consisting of paclitaxel, gemcitabine, trastuzumab, cisplatin, docetaxel, doxorubicin, melphalan and 5-fluorouracil.

The present invention provides a method for treating a patient with a tumor comprising administering to the patient a therapeutically effective amount of a farnesyl protein transferase inhibitor if cells in the tumor underexpress PRL2, claudin-1, mucin-1, LTB4DH or endothelin-1 and/or overexpress PDGFRL, relative to expression of the biomarker by a cell that is resistant to the inhibitor.

The present invention provides a method for treating a patient with a tumor comprising: (a) determining an expression level, by at least one cell in the tumor, of at least one biomarker selected from the group consisting of PDGFRL, PRL2, claudin-1, mucin-1, LTB4DH and endothelin-1; and (b) administering, to the patient, a therapeutically effective amount of a farnesyl protein transferase inhibitor if PRL2, claudin-1, mucin-1, LTB4DH or endothelin-1 is underexpressed relative to its expression by a cell that is resistant to the inhibitor and/or if PDGFRL is overexpressed relative to its expression by a cell that is resistant to the inhibitor.

The present invention provides a method for diagnosing whether a patient with a tumor is likely to respond to therapy with a farnesyl protein transferase inhibitor comprising determining a level of expression by a cell in the tumor of at least one biomarker selected from the group consisting of PDGFRL, PRL2, claudin-1, mucin-1, LTB4DH and endothelin-1; wherein if PRL2, claudin-1, mucin-1, LT84DH or endothelin-1 is underexpressed and/or if PDGFRL and is overexpressed, relative to a cell that is resistant to the inhibitor, then the patient is diagnosed as likely to respond to the inhibitor.

The present invention provides a method for marketing a farnesyl protein transferase inhibitor for treating cancer comprising packaging the inhibitor with a label that recommends use of the inhibitor in a patient having a tumor that underexpresses PRL2, claudin-1, mucin-1, LTB4DH or endothelin-1 and/or overexpresses PDGFRL relative to a cell that is resistant to said inhibitor.

The present invention provides an article of manufacture comprising a farnesyl protein transferase inhibitor and a package insert or label that recommends use of the inhibitor in a patient having a tumor that underexpresses at least one member selected from the group consisting of PRL2, claudin-1, mucin-1, LTB4DH and endothelin-1 and/or overexpresses PDGFRL, relative to a cell that is resistant to said inhibitor.

The present invention provides a screening method to identify tumors responsive to farnesyl protein transferase inhibitors, comprising detecting an amount of a biomarker selected from the group consisting of PDGFRL. PRL2, claudin-1 mucin-1, LTB4DH and endothelin-1 in a cell of said tumor, and identifying the tumor as: (i) a farnesyl protein transferase inhibitor sensitive tumor if the cell underexpresses one or more genes selected from the group consisting of PRL2, claudin-1, mucin-1, LTB4DH and endothelin-1 and/or overexpresses PDGFRL relative to a cell that is resistant to said inhibitor or (ii) a farnesyl protein transferase inhibitor resistant tumor if the cell does not underexpress one or more genes selected from the group consisting of PRL2, claudin-1, mucin-1, LTB4DH and endothelin-1 and/or overexpress PDGFRL relative to a cell that is resistant to said inhibitor.

### BRIEF DESCRIPTION OF THE FIGURES

FIG. 1. Hierarchical clustering using the 98 genes found to be differentially expressed in sensitive vs. resistant cell lines. In this dendrogram, red indicates upregulation relative to the mean and green indicates downregulation. Genes are represented on the x-axis and experiments are on the y-axis. The hierarchical clustering dendrogram was generated using a correlation-based similarity measurement and an average-weighting method.

FIG. 2. RT-PCR analysis of mRNA expression of PRL2, claudin-1, mucin-1, LTB4DH and endothelin-1 and PDGFRL in various cell lines relative to the expression level in a lonafarnib resistant cell line. The breast tissue expression data is relative to expression of the indicated biomarker in cell line T47D. The ovarian tissue expression data is relative to expression of the indicated biomarker in cell line SKOV3 (SKOV). The brain tissue expression data is relative to expression of the indicated biomarker in cell line U87MG. The pancreatic tissue expression data is relative to expression of the indicated biomarker in cell line Aspc1. The leukemic cell expression data is relative to expression of the indicated biomarker in cell line K562. The colon tissue expression data is relative to expression of the indicated biomarker in cell line HT29. The prostate tissue expression data is relative to expression of the indicated biomarker in cell line DU145. Black bars correspond to test cells which were normalized to white bars which correspond to resistant cells.

FIG. 3. (a) Western blot analysis of the level of protein expression of claudin-1, mucin-1 and LTB4DH in six cell lines; (b) ELISA analysis of the level of protein expression of endothelin-1 in six cell lines; (c) cellular levels of PRL1, PRL2 and PRL3 mRNA in cells exposed to PRL2 siRNA (indicated in the legend with an “si prefix”) or control siRNA (indicated in the legend with a “ct” prefix); (d) level of growth inhibition observed in six lonafarnib resistant cell lines exposed to PRL2 siRNA (PRL2 siRNA) or control siRNA (ct siRNA).

### DETAILED DESCRIPTION OF THE INVENTION

The present invention provides methods where by a cancer from which a patient is suffering can be assessed for its responsiveness to an FPT. A cancer can be assessed as FTI resistant or sensitive based on the expression of genes discussed herein either on or in the cancerous cells themselves or as measured in the blood of the patient. Tables 1 and 2 set forth genes whose expression can be assessed. Based on the assessment of a cancer's relative FTI sensitivity or resistance, a clinician or doctor of ordinary skill in the art may make a reasoned decision, based on, e.g., the particular needs of the patient involved and the exigencies of the situation whether to undertake a treatment regimen with an FTI.

The term “patient” or “subject” includes any organism, preferably an animal, more preferably a mammal (e.g., rat, mouse, dog, cat, rabbit) and most preferably a human.

The terms “tumor” or “cell” in said tumor relate to both cells from a solid cancer (e.g., lung cancer) or from a non-solid cancer (e.g., leukemia).

A neoplastic cell is an abnormal cell which divides more than it should and/or does not die when it should.

In an embodiment of the invention, the PRL2 gene is included in the following sequence:

(SEQ ID NO: 1)

agcggggctg cgcgaagtca tcgctgttcc agacagcgat
gactcgagag cggtgggggt ggcggcgcga tcggccgggc
tgtaaccgtc gtctgtccgg gagcggctgg agcggcagcg
gcggccgggc acggcgcgag gtgacgccac agggcagcgg
cggcagcgga ggcagcggcg gcagcaggag acgcagcggc
ggccgcagca gcagcagcaa gacggactcg tggagacgcg
ccgccgccgc cgccgccggg ccgggccggg tgtcgcgcgc
cgaggctggg ggggagtcgt cgccgccgcc gacaccgcta
ccgccgccgc cgccgccgcc gaggtgactg aggagagagg
cgcctcctcg ctcccgccac cgccggactt caatgcccag
tccccagctc gccagcgttt ttcgttggaa tatacgttgc
acatttatgg cgattctgag tgtgagggca gacttctgcc
aggctcagca cagcattttc gctgacaagt gagcttggag
gttctatgtg ccataattaa cattgccttg aagactcctg
gacaccgaga ctggcctcag aaatagttgg cttttttttt
tttttaattg caagcatatt tcttttaatg actccagtaa
aattaagcat caagtaaaca agtggaaagt gacctacact
tttaacttgt ctcactagtg cctaaatgta gtaaaggctg
cttaagtttt gtatgtagtt ggattttttg gagtccgaat
atttccatct gcagaaattg aggcccaaat tgaatttgga
ttcaagtgga ttctaaatac tttgcttatc ttgaagagag
aagcttcata aggaataaac aagttgaata gagaaaacac
tgattgataa taggcatttt agtggtcttt ttaatgtttt
ctgctgtgaa acatttcaag atttattgat tttttttttt
cactttcccc atcacactca cacgcacgct cacacttttt
atttgccata atgaaccgtc cagcccctgt ggagatctcc
tatgagaaca tgcgttttct gataactcac aaccctacca
atgctactct caacaagttc acagaggaac ttaagaagta
tggagtgacg actttggttc gagtttgtga tgctacatat
gataaagctc cagttgaaaa agaaggaatc cacgttctag
attggccatt tgatgatgga gctccacccc ctaatcagat
agtagatgat tggttaaacc tgttaaaaac caaatttcgt
gaagagccag gttgctgtgt tgcagtgcat tgtgttgcag
gattgggaag ggcacctgtg ctggttgcac ttgctttgat
tgaatgtgga atgaagtacg aagatgcagt tcagtttata
agacaaaaaa gaaggggagc gttcaattcc aaacagctgc
tttatttgga gaaataccga cctaagatgc gattacgctt
cagagatacc aatgggcatt gctgtgttca gtagaaggaa
atgtaaacga aggctgactt gattgtgcca tttagaggga
actcttggta cctggaaatg tgaatctgga atattacctg
tgtcatcaaa gtagtgatgg attcagtact cctcaaccac
tctcctaatg attggaacaa aagcaaacaa aaaagaaatc
tctctataaa atgaataaaa tgtttaagaa aagagaaaga
gaaaaggaat taattcagtg aaggatgatt ttgctcctag
ttttggagtt tgaatttctg ccaggattga attattttga
aatctcctgt ctttttaaac tttttcaaaa taggtctcta
aggaaaacca gcagaacatt aggcctgtgc aaaaccatct
gtttggggag cacactcttc cattatgctt ggcacataga
tctccctgtg gtgggatttt ttttttccct ttttttgtgg
gggagggttg gtggtatatt tttcccctct tttttccttc
ctctcctaca tctccctttt cccccgatcc aagttgtaga
tggaatagaa gcccttgttg ctgtagatgt gcgtgcagtc
tggcagcctt aagcccacct gggcactttt agataaaaaa
aaaaaaaaac aaaaaacaac accaaaaaaa cagcagtgat
atatatattc caggtggttt ttagtcttta ctgatgaaag
ggtgttcatg ttagtttctt caaaacccta tctaatacta
ggcaaagtag ccaagagcct tttgttttgt ttttattttg
ataaattagt ggagaaatgg cattttaaga ggagtctctt
ctcaacttac ctgagagtcg aattcttctc ttccctaacc
aatgaagcta agtggttatc ccagaaactt gtcttctaaa
agggaggact ccaggccatc aataaagatg tccaggcagt
gagcgcactt tttacaccct gtagaattgt gggctgtagc
gttactctga ttttctgtct agtatcagag aatgctggta
gcttaaaatt tttattttag gacttgtact ctgaattttc
aggaaccgtc aaaggagcag cagcaaattc acatattttc
gacttgagaa atgcttgtgg tatgtgtttt ccaaactgcc
ccctatatgt aaagttcagt ttaaccactg attgccttgt
tattactagg ttttttgaga ttaaaaaaaa aaaatccctg
gtttaaaacc aacaatgatg cctagtgagt atgtgtccac
aggccataac agggtagaag agagacatcg tgcaacccaa
tgagtagtga agggactgtg ttgcttgtga agcggtgtag
tagcattttt gcagattctt ggctgggtct agtgtactga
tctagaaaag ctgtttttct gctcctttgt ggaaggcagt
tatgatcagg ctgcatggac aaagcaggta gaggggcacc
atcaggggct cttgcactat tttcacctct aaatattacg
tactcagtag tgccctgctt ctagggctct gaatacgggc
ttaaagtcat cttgtcctgc tggaatttgc tgtgcagagc
cataagcctc ccattttgtt agcgtcagct aggccaatag
gaacagaccg ggaccttgtc tcacactgat gatacctcac
atgttgaccg gctatgtgaa ctgcctattt cctatgctgg
agttttgatt tttaactaaa cgcaaatctg tagattctct
cctctcccat cccagaaaac aaaacaaaat aatgcttttc
gaaattgttt ctaggacttt aaaacataat ggtatatcca
aaattcttta tttcagaatg caacaataga ttccattaat
atagactcaa gatcaaaaca gtatacctgc taagctaaga
tagatggtgt tgattccact gggttttgat caatacaata
acaaaccttt ttcctttgac atactctgaa ttttgttgtt
tggggggagg gggtgtgtgt gtgtgtgtgt gtgtgtgtgt
gtattgtgtg tgtgtgtgtg tgcacgcgca gtgtccatca
gtatcagtgc ctgcctgagt taggaaaatt acattcctgg
ttctgtattg aggagaagga tgtataaagc aacatgaaac
attagccttt cttttatttt aaagactatt gttaattgtt
cttaaaactg gatttttttt ccttaaagca atttttttct
tttcgattta atgaagtatt gctagctgaa gccagtttga
catagagaga tgtcagattg atttgaaagg tgtgcagcct
gattcaaaac caaaccctga acccttttaa agaacaataa
aacatatttt acacgctcaa aaaaaaaaaa aaaaaaaaaa
aaaaaaaaaa aaaaaaaaaa aaaaaaaaaa aaaaaaaaaa
aaaaa

wherein the PRL2 open reading frame thereof comprises the nucleotide sequence:

(SEQ ID NO: 2)

atgaaccgtc cagcccctgt ggagatctcc tatgagaaca
tgcgttttct gataactcac aaccctacca atgctactct
caacaagttc acagaggaac ttaagaagta tggagtgacg
actttggttc gagtttgtga tgctacatat gataaagctc
cagttgaaaa agaaggaatc cacgttctag attggccatt
tgatgatgga gctccacccc ctaatcagat agtagatgat
tggttaaacc tgttaaaaac caaatttcgt gaagagccag
gttgctgtgt tgcagtgcat tgtgttgcag gattgggaag
ggcacctgtg ctggttgcac ttgctttgat tgaatgtgga
atgaagtacg aagatgcagt tcagtttata agacaaaaaa
gaaggggagc gttcaattcc aaacagctgc tttatttgga
gaaataccga cctaagatgc gattacgctt cagagatacc
aatgggcatt gctgtgttca gtag

In an embodiment of the invention, the PRL2 gene encodes:

(SEQ ID NO: 3)

MNRPAPVEISYENMRFLITHNPTNATLNKFTEELKKYGVTTLVRVCDATY
DKAPVEKEGIHVLDWPFDDGAPPPNQIVDDWLNLLKTKFREEPGCCVAVH
CVAGLGRAPVLVALALIECGMKYEDAVQFIRQKRRGAFNSKQLLYLEKYR
PKMRLRFRDTNGHCCVQ

See also Rommens et al. Genomics 28 (3): 530-542 (1995); Montagna et al. Hum. Genet. 96 (5), 532-538 (1995); Zhao et al., Genomics 35 (1), 172-181 (1996); or Genbank accession no. NM_{—}003479.

PRL2 is a prenylation dependent protein-tyrosine phosphatase which is prenylated by farnesyl protein transferase (Zeng et al., J. Bio. Chem. 275(28): 21444-21452; Basso et al., J. Lipid. Res. (2006) 47; 15-31; Wang et al., J. Biol. Chem. (2002) 277(48):46659-68).

Claudins are integral membrane proteins that, along with occluding and junctional adhesion molecules, form tight junctions between cells. Tumors have been shown to have altered claudin expression when compared to that of normal surrounding tissue.

In an embodiment of the invention, claudin-1 comprises the amino acid sequence:

(SEQ ID NO: 40)

MANAGLQLLGFILAFLGWIGAIVSTALPQWRIYSYAGDNIVTAQAMYEGL
WMSCVSQSTGQIQCKVFDSLLNLSSTLQATRALMVVGILLGVIAIFVATV
GMKCMKCLEDDEVQKMRMAVIGGAIFLLAGLAILVATAWYGNRIVQEFYD
PMTPVNARYEFGQALFTGWAAASLCLLGGALLCCSCPRKTTSYPTPRPYP
KPAPSSGKDYV

and the claudin-1 polynucleotide comprises the sequence (open reading frame of claudin-1 is nucleotides 221-856):

(SEQ ID NO: 30)

gagcaaccgcagcttctagtatccagactccagcgccgccccgggcgcgg
accccaaccccgacccagagcttctccagcggcggcgcagcgagcagggc
tccccgccttaacttcctccgcggggcccagccaccttcgggagtccggg
ttgcccacctgcaaactctccgccttctgcacctgccacccctgagccag
cgcgggcgcccgagcgagtcatggccaacgcggggctgcagctgttgggc
ttcattctcgccttcctgggatggatcggcgccatcgtcagcactgccct
gccccagtggaggatttactcctatgccggcgacaacatcgtgaccgccc
aggccatgtacgaggggctgtggatgtcctgcgtgtcgcagagcaccggg
cagatccagtgcaaagtctttgactccttgctgaatctgagcagcacatt
gcaagcaacccgtgccttgatggtggttggcatcctcctgggagtgatag
caatctttgtggccaccgttggcatgaagtgtatgaagtgcttggaagac
gatgaggtgcagaagatgaggatggctgtcattgggggtgcgatatttct
tcttgcaggtctggctattttagttgccacagcatggtatggcaatagaa
tcgttcaagaattctatgaccctatgaccccagtcaatgccaggtacgaa
tttggtcaggctctcttcactggctgggctgctgcttctctctgccttct
gggaggtgccctactttgctgttcctgtccccgaaaaacaacctcttacc
caacaccaaggccctatccaaaacctgcaccttccagcgggaaagactac
gtgtgacacagaggcaaaaggagaaaatcatgttgaaacaaaccgaaaat
ggacattgagatactatcattaacattaggaccttagaattttgggtatt
gtaatctgaagtatggtattacaaaacaaacaaacaaacaaaaaacccat
gtgttaaaatactcagtgctaaacatggcttaatcttattttatcttctt
tcctcaatataggagggaagatttttccatttgtattactgcttcccatt
gagtaatcatactcaattgggggaaggggtgctccttaaatatatataga
tatgtatatatacatgtttttctattaaaaatagacagtaaaatactatt
ctcattatgttgatactagcatacttaaaatatctctaaaataggtaaat
gtatttaattccatattgatgaagatgtttattggtatattttctttttc
gtctatatatacatatgtaacagtcaaatatcatttactcttcttcatta
gctttgggtgcctttgccacaagacctagcctaatttaccaaggatgaat
tctttcaattcttcatgcgtgcccttttcatatacttattttatttttta
ccataatcttatagcacttgcatcgttattaagcccttatttgttttgtg
tttcattggtctctatctcctgaatctaacacatttcatagcctacattt
tagtttctaaagccaagaagaatttattacaaatcagaactttggaggca
aatctttctgcatgaccaaagtgataaattcctgctgaccttcccacaca
atccctgtactctgacccatagcactcttgtttgctttgaaaatatttgt
ccaattgagtagctgcatgctgttcccccaggtgttgtaacacaacttta
ttgattgaatttttaagctacttattcatagttttatatccccctaaact
acctttttgttccccattccttaattgtattgttttcccaagtgtaatta
tcatgcgttttatatcttcctaataaggtgtggtctgtttgtctgaacaa
agtgctagactttctggagtgataatctggtgacaaatattctctctgta
gctgtaagcaagtcacttaatctttctacctcttttttctatctgccaaa
ttgagataatgatacttaaccagttagaagaggtagtgtgaatattaatt
agtttatattactctcattctttgaacatgaactatgcctatgtagtgtc
tttatttgctcagctggctgagacactgaagaagtcactgaacaaaacct
acacacgtaccttcatgtgattcactgccttcctctctctaccagtctat
ttccactgaacaaaacctacacacataccttcatgtggttcagtgccttc
ctctctctaccagtctatttccactgaacaaaacctacgcacataccttc
atgtggctcagtgccttcctctctctaccagtctatttccattctttcag
ctgtgtctgacatgtttgtgctctgttccattttaacaactgctcttact
tttccagtctgtacagaatgctatttcacttgagcaagatgatgtaatgg
aaagggtgttggcattggtgtctggagacctggatttgagtcttggtgct
atcaatcaccgtctgtgtttgagcaaggcatttggctgctgtaagcttat
tgcttcatctgtaagcggtggtttgtaattcctgatcttcccacatcaca
gtgatgttgtggggatccagtgagatagaatacatgtaagtgtggttttg
taatttaaaaagtgctatactaagggaaagaattgaggaattaactgcat
acgttttggtgttgcttttcaaatgtttgaaaacaaaaaaaatgttaaga
aatgggtttcttgccttaaccagtctctcaagtgatgagacagtgaagta
aaattgagtgcactaaacaaataagattctgaggaagtcttatcttctgc
agtgagtatggcccgatgctttctgtggctaaacagatgtaatgggaaga
aataaaagcctacgtgttggtaaatccaacagcaagggagatttttgaat
cataataactcataaggtgctatctgttcagtgatgccctcagagctctt
gctgttagctggcagctgacgctgctaggatagttagtttggaaatggta
cttcataataaactacacaaggaaagtcagccactgtgtcttatgaggaa
ttggacctaataaattttagtgtgccttccaaacctgagaatatatgctt
ttggaagttaaaatttaaatggcttttgccacatacatagatcttcatga
tgtgtgagtgtaattccatgtggatatcagttaccaaacattacaaaaaa
attttatggcccaaaatgaccaacgaaattgttacaatagaatttatcca
attttgatctttttatattcttctaccacacctggaaacagaccaataga
cattttggggttttataataggaatttgtataaagcattactctttttca
ataaattgttttttaatttaaaaaaaggaaaaaaaaaaaaaaaaa

Leukotriene B4 12-hydroxydehydrogenase (LTB4DH) inhibits the pro-inflammatory actions of LTB4. Differential expression analysis previously identified LTB4DH as a gene upregulated by dithiolthiones, which are known to inhibit tumorigenesis in preclinical models.

In an embodiment of the invention, LTB4DH comprises the amino acid sequence:

(SEQ ID NO: 42)

MVRTKTWTLKKHFVGYPTNSDFELKTSELPPLKNGEVLLEALFLTVDPYM
RVAAKRLKEGDTMMGQQVAKVVESKNVALPKGTIVLASPGWTTHSISDGK
DLEKLLTEWPDTIPLSLALGTVGMPGLTAYFGLLEICGVKGGETVMVNAA
AGAVGSVVGQIAKLKGCKVVGAVGSDEKVAYLQKLGFDVVFNYKTVESLE
ETLKKASPDGYDCYFDNVGGEFSNTVIGQMKKFGRIAICGAISTYNRTGP
LPPGPPPEIVIYQELRMEAFVVYRWQGDARQKALKDLLKWVLEGKIQYKE
YIIEGFENMPAAFMGMLKGDNLGKTIVKA

and the LTB4DH polynucleotide comprises the sequence (open reading frame of LTB4DH is nucleotides 104-1093):

(SEQ ID NO: 41)

gtcccgacgcctcccgcccccgcagttccttggagagcttggagccgcgc
gccggagggaataggaaagcttggttacaacccgggacacccggagcttc
aggatggttcgtactaagacatggaccctgaagaagcactttgttggcta
tcctactaatagtgactttgagttgaagacatctgagctcccacccttaa
aaaatggagaggtcctgcttgaagctttgttcctcaccgtggatccctac
atgagagtggcagccaaaagattgaaggaaggtgatacaatgatggggca
gcaagtggccaaagttgtggaaagtaaaaatgtagccctaccaaaaggaa
ctattgtactggcttctccaggctggacaacgcactccatttctgatggg
aaagatctggaaaagctgctgacagagtggccagacacaataccactgtc
tttggctctggggacagttggcatgccaggcctgactgcctactttggcc
tacttgaaatctgtggtgtgaagggtggagaaacagtgatggttaatgca
gcagctggagctgtgggctcagtcgtggggcagattgcaaagctcaaggg
ctgcaaagttgttggagcagtagggtctgatgaaaaggttgcctaccttc
aaaagcttggatttgatgtcgtctttaactacaagacggtagagtctttg
gaagaaaccttgaagaaagcgtctcctgatggttatgattgttattttga
taatgtaggtggagagttttcaaacactgttatcggccagatgaagaaat
ttggaaggattgccatatgtggagccatctctacatataacagaaccggc
ccacttcccccaggcccacccccagagattgttatctatcaggagcttcg
catggaagcttttgtcgtctaccgctggcaaggagatgcccgccaaaaag
ctctgaaggacttgctgaaatgggtcttagagggtaaaatccagtacaag
gaatatatcattgaaggatttgaaaacatgccagccgcatttatggaaat
gctgaaaggagataatttggggaagacaatagtgaaagcatgaaaaagag
gacacatggaatctggaggccatttagatgattagttaatttgtttttca
ccatttagcaaaaatgtatactaccttaaatgtcttaagaaatagtactc
ataatgagtttgagctacttaataaaatacatttaagtggtaaaaaaaaa
aaaaaaa

Mucin-1 is a transmembrane glycoprotein expressed on the apical border of cells. The gene is believed to lubricate the passage of material and protect the epithelial lining. Mucin-1 is overexpressed, aberrantly glycosylated, or expressed over the entire cell surface in tumor cells.

In an embodiment of the invention, mucin-1 comprises the amino acid sequence:

(SEQ ID NO: 44)

MTPGTQSPFFLLLLLTVLTVVTGSGHASSTPGGEKETSATQRSSVPSSTE
KNALSTGVSFFFLSFHISNLQFNSSLEDPSTDYYQELQRDISEMFLQIYK
QGGFLGLSNIKFRPGSVVVQLTLAFREGTINVHDVETQFNQYKTEAASRY
NLTISDVSVSDVPFPFSAQSGAGVPGWGIALLVLVCVLVALAIVYLIALA
VCQCRRKNYGQLDIFPARDTYHPMSEYPTYHTHGRYVPPSSTDRSPYEKV
SAGNGGSSLSYTNPAVAATSANL

and the mucin-1 polynucleotide comprises the sequence (open reading frame of mucin-1 is nucleotides 67-888):

(SEQ ID NO: 43)

acctctcaagcagccagcgcctgcctgaatctgttctgccccctccccac
ccatttcaccaccaccatgacaccgggcacccagtctcctttcttcctgc
tgctgctcctcacagtgcttacagttgttacgggttctggtcatgcaagc
tctaccccaggtggagaaaaggagacttcggctacccagagaagttcagt
gcccagctctactgagaagaatgctttgtctactggggtctctttctttt
tcctgtcttttcacatttcaaacctccagtttaattcctctctggaagat
cccagcaccgactactaccaagagctgcagagagacatttctgaaatgtt
tttgcagatttataaacaagggggttttctgggcctctccaatattaagt
tcaggccaggatctgtggtggtacaattgactctggccttccgagaaggt
accatcaatgtccacgacgtggagacacagttcaatcagtataaaacgga
agcagcctctcgatataacctgacgatctcagacgtcagcgtgagtgatg
tgccatttcctttctctgcccagtctggggctggggtgccaggctggggc
atcgcgctgctggtgctggtctgtgttctggttgcgctggccattgtcta
tctcattgccttggctgtctgtcagtgccgccgaaagaactacgggcagc
tggacatctttccagcccgggatacctaccatcctatgagcgagtacccc
acctaccacacccatgggcgctatgtgccccctagcagtaccgatcgtag
cccctatgagaaggtttctgcaggtaatggtggcagcagcctctcttaca
caaacccagcagtggcagccacttctgccaacttgtaggggcacgtcgcc
cgctgagctgagtggccagccagtgccattccactccactcaggttcttc
agggccagagcccctgcaccctgtttgggctggtgagctgggagttcagg
tgggctgctcacagcctccttcagaggccccaccaatttctcggacactt
ctcagtgtgtggaagctcatgtgggcccctgagggctcatgcctgggaag
tgttgtggtgggggctcccaggaggactggcccagagagccctgagatag
cggggatcctgaactggactgaataaaacgtggtctcccactgcgccaaa
aaaaaaaaa

Endothelins (ETs) are a family vasoconstrictor peptides. Endothelin-1 has been shown to induce the proliferation of certain cancerous cells. Endothelin-1 is soluble blood protein Endothelin-1 in the blood of a patient, or any fraction thereof (e.g., serum or plasma), can be assayed in order to assess the FTI sensitivity of any cancer from which the patient suffers. A high level of endothelin-1 in the blood of a patient (or a fraction thereof) indicates that the cancer from which the patient suffers is FTI resistant. In an embodiment of the invention, endothelin-1 comprises the amino acid sequence:

(SEQ ID NO: 46)

MDYLLMIFSLLFVACQGAPETAVLGAELSAVGENGGEKPTPSPPWRLRRS
KRCSCSSLMDKECVYFCHLDIIWVNTPEHVVPYGLGSPRSKRALENLLPT
KATDRENRCQCASQKDKKCWNFCQAGKELRAEDIMEKDWNNHKKGKDCSK
LGKKCIYQQLVRGRKIRRSSEEHLRQTRSETMRNSVKSSFHDPKLKGNPS
RERYVTHNRAHW

and the endothelin-1 polynucleotide comprises the sequence (open reading frame of endothelin-1 is nucleotides 204-842):

(SEQ ID NO: 45)

cgccgcgtgcgcctgcagacgctccgctcgctgccttctctcctggcagg
cgctgccttttctccccgttaaagggcacttgggctgaaggatcgctttg
agatctgaggaacccgcagcgctttgagggacctgaagctgtttttcttc
gttttcctttgggttcagtttgaacgggaggtttttgatccctttttttc
agaatggattatttgctcatgattttctctctgctgtttgtggcttgcca
aggagctccagaaacagcagtcttaggcgctgagctcagcgcggtgggtg
agaacggcggggagaaacccactcccagtccaccctggcggctccgccgg
tccaagcgctgctcctgctcgtccctgatggataaagagtgtgtctactt
ctgccacctggacatcatttgggtcaacactcccgagcacgttgttccgt
atggacttggaagccctaggtccaagagagccttggagaatttacttccc
acaaaggcaacagaccgtgagaatagatgccaatgtgctagccaaaaaga
caagaagtgctggaatttttgccaagcaggaaaagaactcagggctgaag
acattatggagaaagactggaataatcataagaaaggaaaagactgttcc
aagcttgggaaaaagtgtatttatcagcagttagtgagaggaagaaaaat
cagaagaagttcagaggaacacctaagacaaaccaggtcggagaccatga
gaaacagcgtcaaatcatcttttcatgatcccaagctgaaaggaaatccc
tccagagagcgttatgtgacccacaaccgagcacattggtgacagacctt
cggggcctgtctgaagccatagcctccacggagagccctgtggccgactc
tgcactctccaccctggctgggatcagagcaggagcatcctctgctggtt
cctgactggcaaaggaccagcgtcctcgttcaaaacattccaagaaaggt
taaggagttcccccaaccatcttcactggcttccatcagtggtaactgct
ttggtctcttctttcatctggggatgacaatggacctctcagcagaaaca
cacagtcacattcgaattcgggtggcatcctccggagagagagagaggaa
ggagattccacacaggggtggagtttctgacgaaggtcctaagggagtgt
ttgtgtctgactcaggcgcctggcacatttcagggagaaactccaaagtc
cacacaaagattttctaaggaatgcacaaattgaaaacacactcaaaaga
caaacatgcaagtaaagaaaaaaaaaaaaaaaaa

PDGFRL is the platelet-derived growth factor receptor-like protein precursor which bears significant sequence similarity to the ligand binding domain of platelet-derived growth factor receptor beta. PDGFRL has been shown to have tumor suppressor activity.

In an embodiment of the invention, PDGFRL comprises the amino acid sequence:

(SEQ ID NO: 48)

MKVWLLLGLLLVHEALEDVTGQHLPKNKRPKEPGENRIKPTNKKVKPKIP
KMKDRDSANSAPKTQSIMMQVLDKGRFQKPAATLSLLAGQTVELRCKGSR
IGWSYPAYLDTFKDSRLSVKQNERYGQLTLVNSTSADTGEFSCWVQLCSG
YICRKDEAKTGSTYIFFTEKGELFVPSPSYFDVVYLNPDRQAVVPCRVTV
LSAKVTLHREFPAKEIPANGTDIVYDMKRGFVYLQPHSEHQGVVYCRAEA
GGRSQISVKYQLLYVAVPSGPPSTTILASSNKVKSGDDISVLCTVLGEPD
VEVEFTWIFPGQKDERPVTIQDTWRLIHRGLGHTTRISQSVITVEDFETI
DAGYYICTAQNLQGQTTVATTVEFS

and the PDGFRL polynucleotide comprises the sequence (open reading frame of PDGRRL is nucleotides 62-189):

(SEQ ID NO: 47)

cctgcgtccccgccccgcgcagccgccgcgctcctgcgctccgaggtccg
aggttcccgagatgaaggtctggctgctgcttggtcttctgctggtgcac
gaagcgctggaggatgttactggccaacaccttcccaagaacaagcgtcc
aaaagaaccaggagagaatagaatcaaacctaccaacaagaaggtgaagc
ccaaaattcctaaaatgaaggacagggactcagccaattcagcaccaaag
acgcagtctatcatgatgcaagtgctggataaaggtcgcttccagaaacc
cgccgctaccctgagtctgctggcggggcaaactgtagagcttcgatgta
aagggagtagaattgggtggagctaccctgcgtatctggacacctttaag
gattctcgcctcagcgtcaagcagaatgagcgctacggccagttgactct
ggtcaactccacctcggcagacacaggtgaattcagctgctgggtgcagc
tctgcagcggctacatctgcaggaaggacgaggccaaaacgggctccacc
tacatcttttttacagagaaaggagaactctttgtaccttctcccagcta
cttcgatgttgtctacttgaacccggacagacaggctgtggttccttgtc
gggtgaccgtgctgtcggccaaagtcacgctccacagggaattcccagcc
aaggagatcccagccaatggaacggacattgtttatgacatgaagcgggg
ctttgtgtatctgcaacctcattccgagcaccagggtgtggtttactgca
gggcggaggccgggggcagatctcagatctccgtcaagtaccagctgctc
tacgtggcggttcccagtggccctccctcaacaaccatcttggcttcttc
aaacaaagtgaaaagtggggacgacatcagtgtgctctgcactgtcctgg
gggagcccgatgtggaggtggagttcacctggatcttcccagggcagaag
gatgaaaggcctgtgacgatccaagacacttggaggttgatccacagagg
actgggacacaccacgagaatctcccagagtgtcattacagtggaagact
tcgagacgattgatgcaggatattacatttgcactgctcagaatcttcaa
ggacagaccacagtagctaccactgttgagttttcctgacttggaaaagg
aaatgtaatgaacttatggaaagcccatttgtgtacacagtcagctttgg
ggttccttttattagtgctttgccagaggctgatgtcaagcaccacaccc
caaccccagcgtctcgtgagtccgacccagacatccaaactaaaaggaag
tcatccagtctattcacagaagtgttaacttttctaacagaaagcatgat
tttgattgcttacctacatacgtgttcctagtttttatacatgtgtaaac
aattttatataatcaatcatttctattaaatgagcacgtttttgtaaaaa
at

The present invention comprises embodiments wherein any of the biomarkers set forth herein (e.g., table 1 or 2) are underexpressed or overexpressed to any degree relative to a FPT inhibitor (e.g., lonafarnib) resistant cell line. In an embodiment of the invention, the degree of overexpression or underexpression is approximately as set forth in table 1 (e.g., PRL2, claudin-1, mucin-1, LTB4DH or endothelin-1) or 2 (e.g., PDGFRL) (e.g., in an embodiment of the invention ±0.5%, ±1%, ±2%, ±3, ±4, ±5%, ±10%, ±15% or ±20% relative to a resistant cell line). In an embodiment of the invention, a cell (e.g., in a tumor) that underexpresses a gene selected from table 1 (e.g., PRL2, claudin-1, mucin-1, LTB4DH or endothelin-1) or overexpresses a gene selected from table 2 (e.g., PDGFRL) by an amount at least about 2.5 fold less or more, respectively, than that of a cell resistant to FTIs (e.g., lonafarnib) is considered FTI sensitive.

Overexpression or underexpression of a biomarker in a cell is relative to that of a cell which is resistant to any FPT inhibitor such as lonafarnib. A resistant cell includes any cell whose growth of survival is not significantly reduced by exposure to a given farnesyl protein transferase inhibitor. In an embodiment of the invention, a resistant cell is T47D, SKOV3, SNB75, U-87MG, ASPC1, K562, HT29 or DU145 or any cell, for example, which is known in the art, that exhibits at least as much FTI resistance of these cells. T47D is a human breast cancer cell line available from the American Type Culture Collection (ATCC) under accession number HTB-133. SKOV3 is a human ovary adenocarcinoma cell line also available from ATCC under accession number HTB-77. In an embodiment of the invention, a farnesyl protein transferase inhibitor resistant cell, for example, exhibiting resistance to lonafarnib, exhibits an IC50 of 1000 nM or more. U-87MG is a cell derived from malignant gliomas available from ATCC under accession number HTB-14. ASPC-1 is a cell line derived from nude mouse xenografts initiated with cells from the ascites of a patient with cancer of the pancreas available from ATCC under accession number CRL-1682. HT-29 is a cell line isolated from a primary colorectal adenocarcinoma tumor available from ATCC under accession number HTB-38. The DU145 cell line was isolated from a lesion in the brain of a patient with metastatic carcinoma of the prostate and a 3 year history of lymphocytic leukemia available from ATCC under accession number HTB-81.

In an embodiment of the invention, a cell is sensitive or responsive to a farnesyl protein transferase inhibitor if its growth or survival or ability to metastasize is reduced to any detectable degree. An embodiment of the invention, a cell is sensitive if the IC50 for an inhibitor is less than 1000 nM (e.g., 750 nM, 500 nM, 100 nM, 50 nM, 25 nM, 1 nM, 2 nM, or 3 nM or less).

### Farnesyl Protein Transferase Inhibitors (FTIs)

The present invention includes methods comprising the use of any farnesyl protein transferase inhibitor known in the art. In an embodiment of the invention, the FPT inhibitor (FTI) is one or more of any of the following. 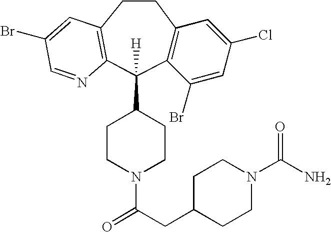
(lonafarnib; Sarasar™; Schering Corp.; Kenilworth, N.J.; see U.S. Pat. Nos. 5,874,442 and 5,719,148).

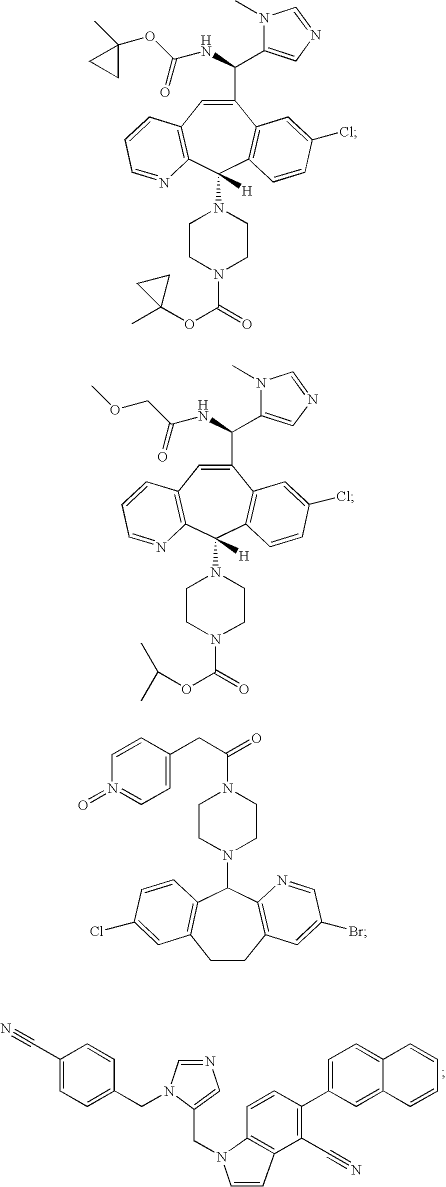

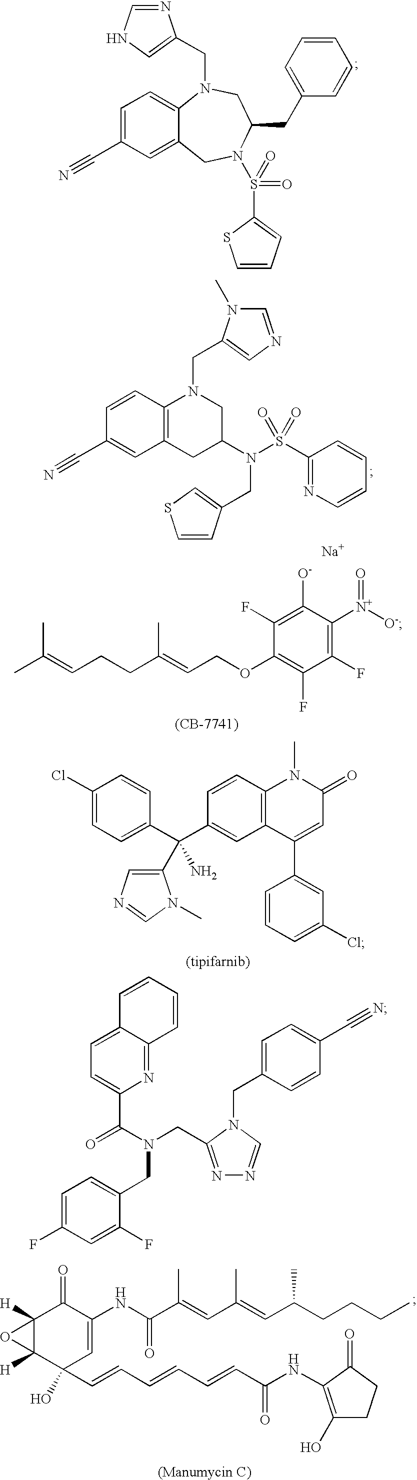

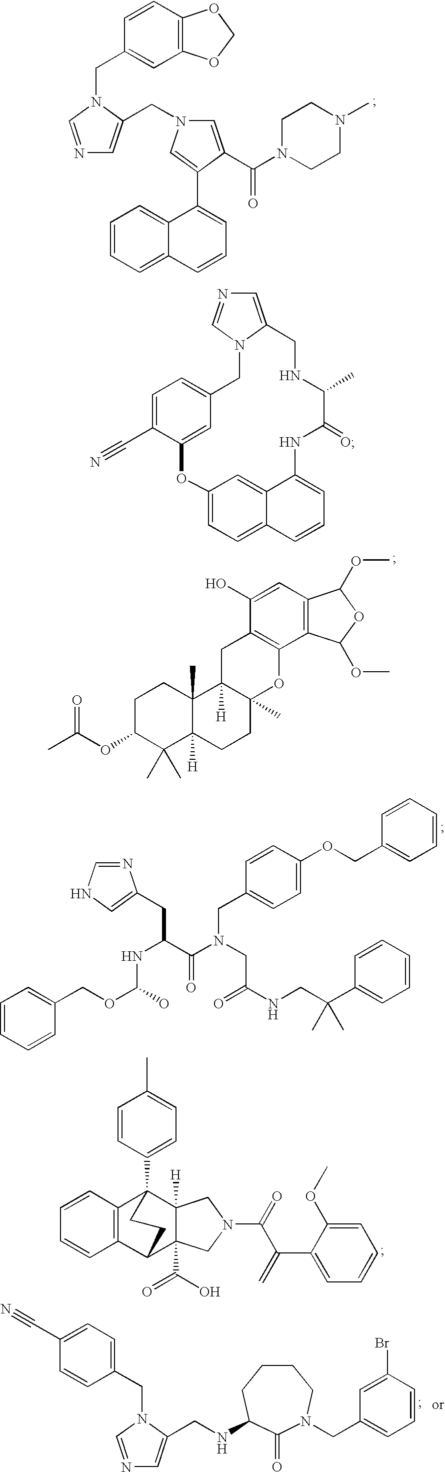

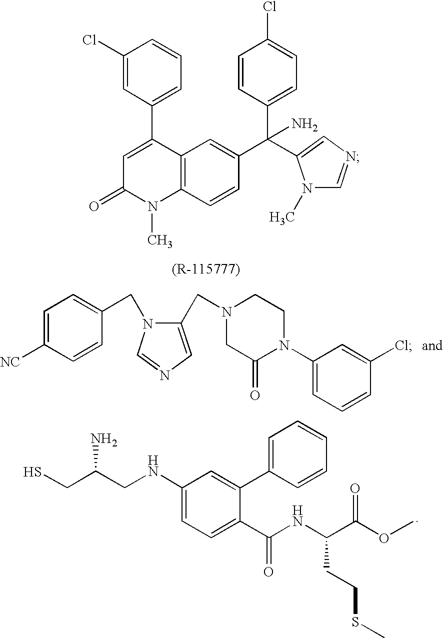

### Other Chemotherapeutic Agents

The present invention comprise methods wherein a farnesyl protein transferase inhibitor is administered to a subject in association with a therapeutic procedure (e.g., surgical tumorectomy or anti-cancer radiation therapy) and/or a further chemotherapeutic agent, such as any anti-cancer chemotherapeutic agent.

In an embodiment of the invention, an FPT inhibitor is provided in association with etoposide (VP-16;

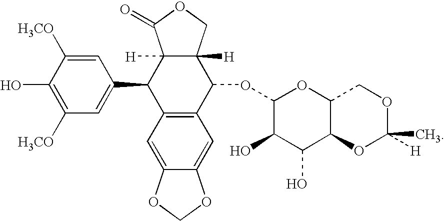

In an embodiment of the invention, an FPT inhibitor is provided in association with gemcitabine

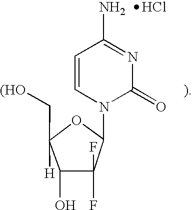

In an embodiment of the invention, an FPT inhibitor is provided in association with any compound disclosed in published U.S. patent application no. U.S. 2004/0209878A1 (e.g., comprising a core structure represented by

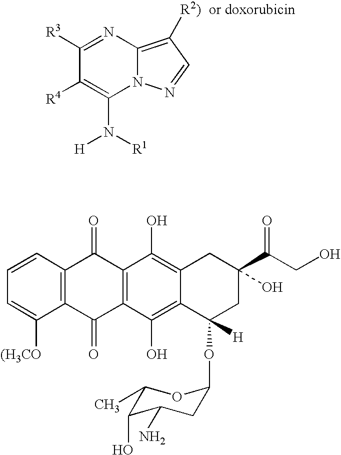

) including Caelyx or Doxil® (doxorubicin HCl liposome injection; Ortho Biotech Products L. P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, an FPT inhibitor is provided in association with 5′-deoxy-5-fluorouridine

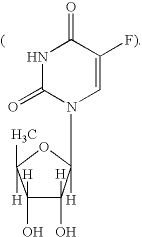

In an embodiment of the invention, an FPT inhibitor is provided in association with vincristine (

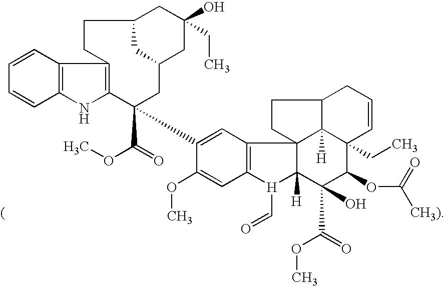

In an embodiment of the invention, an FPT inhibitor is provided in association with temozolomide 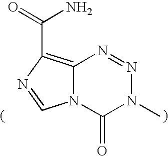
any CDK inhibitor such as ZK-304709, Seliciclib (R-roscovitine) 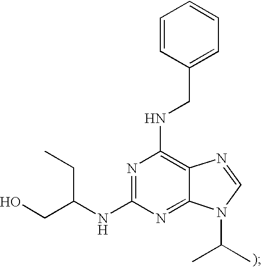
any MEK inhibitor such as PD0325901 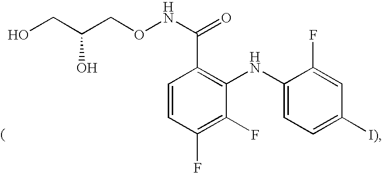
AZD-6244; capecitabine (5′-deoxy-5-fluoro-N-[(pentyloxy) carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate 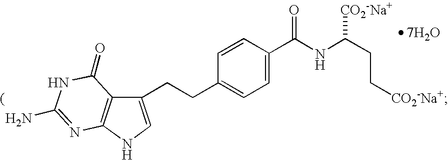
; Pemetrexed disodium heptahydrate).

In an embodiment of the invention, an FPT inhibitor is provided in association with camptothecin 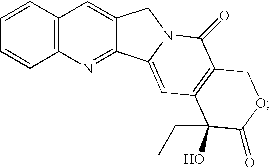
Stork et al., J. Am. Chem. Soc. 93(16): 4074-4075 (1971); Beisler et al., J. Med. Chem. 14(11): 1116-1117 (1962)) or irinotecan ( 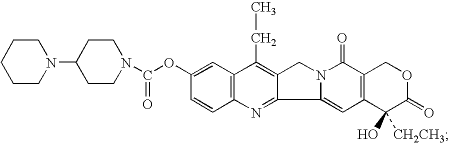
sold as Camptosar®; Pharmacia & Upjohn Co.; Kalamazoo, Mich.).

In an embodiment of the invention, an FPT inhibitor is provided in association with the FOLFOX regimen (oxaliplatin 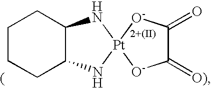
together with infusional fluorouracil 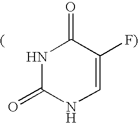
and folinic acid 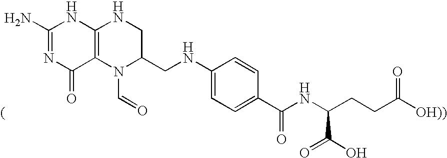
(Chaouche et al. Am. J. Clin. Oncol. 23(3):288-289 (2000), de Gramont et al., J. Clin. Oncol. 18(16):2938-2947 (2000)).

In an embodiment of the invention, an FPT inhibitor is provided in association with melphalan

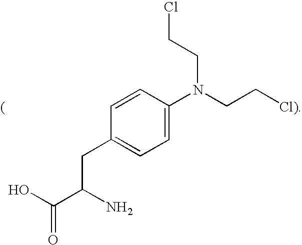

In an embodiment of the invention, an FPT inhibitor is provided in association with an anti-estrogen such as 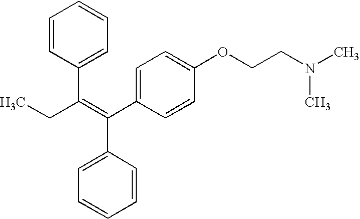
(tamoxifen; sold as Nolvadex® by AstraZeneca Pharmaceuticals LP: Wilmington Del.) or 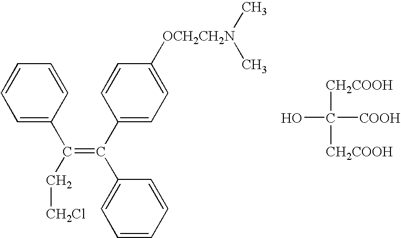
(toremifene citrate; sold as Fareston® by Shire US, Inc.; Florence, Ky.).

In an embodiment of the invention, an FPT inhibitor is provided in association with an aromatase inhibitor such as 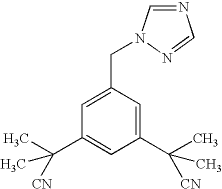
(anastrazole; sold as Arimidex® by AstraZeneca Pharmaceuticals LP; Wilmington, Del.), 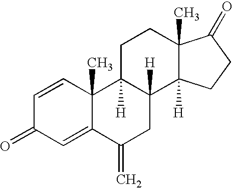
(exemestane; sold as Aromasin® by Pharmacia Corporation; Kalamazoo, Mich.) or 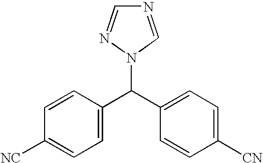
(letrozole; sold as Femara® by Novartis Pharmaceuticals Corporation; East Hanover N.J.).

In an embodiment of the invention, an FPT inhibitor is provided in association with an estrogen such as DES (diethylstilbestrol), 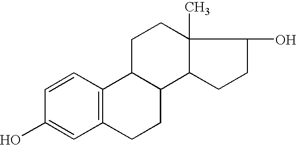
(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, an FPT inhibitor is provided in association with anti-angiogenesis agents including bevacizumab (Avastin™; Genentech; San Francisco, Calif.), the anti-VEGFR-2 antibody IMC-1C11, other VEGFR inhibitors including, but not limited to, CHIR-258 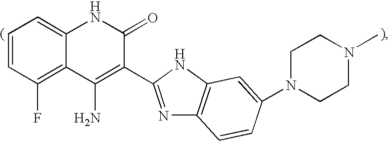
any of the inhibitors set forth in WO2004/13145 (e.g., comprising the core structural formula: 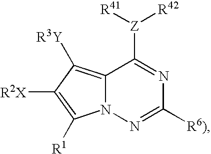
WO2004/09542 (e.g., comprising the core structural formula 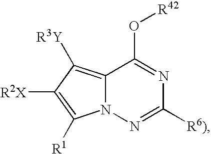
WO00/71129 (e.g., comprising the core structural formula: 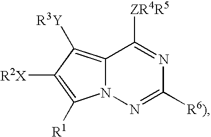
WO2004/09601 (e.g., comprising the core structural formula: 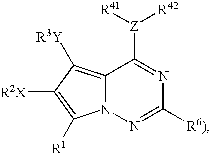
WO2004/01059 (e.g., comprising the core structural formula: 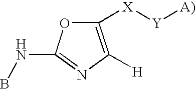
WO01/29025 (e.g., comprising the core structural formula: 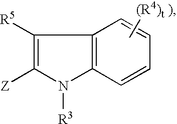
WO02/32861 (e.g., comprising the core structural formula: 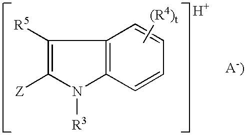
or set forth in WO03/88900 (e.g., comprising the core structural formula 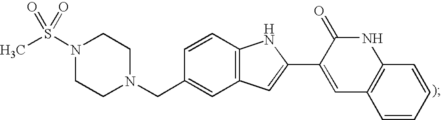
3-[5-(methylsulfonylpiperadinemethyl)-indolyl]-quinolone; Vatalanib 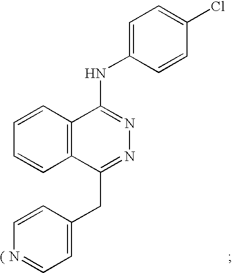
PTK/ZK; CPG-79787; ZK-222584), AG-013736 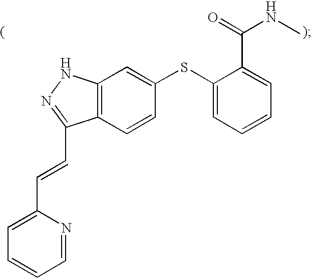
and the VEGF trap (AVE-0005), a soluble decoy receptor comprising portions of VEGF receptors 1 and 2.

In an embodiment of the invention, an FPT inhibitor is provided in association with a LHRH (Lutenizing hormone-releasing hormone) agonist such as the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4]; 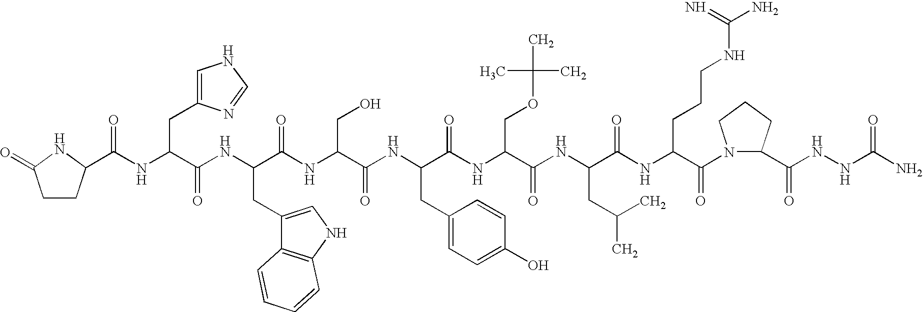
(goserelin acetate; sold as Zoladex® by AstraZeneca UK Limited, Macclesfield, England), 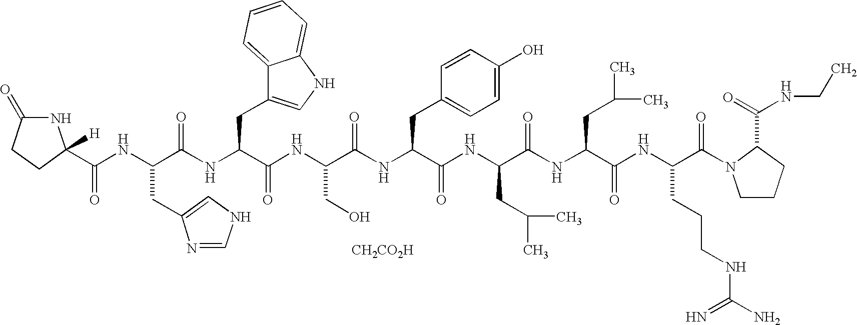
(leuprolide acetate; sold as Eligard® by Sanofi-Synthelabo Inc.; New York, N.Y.) or 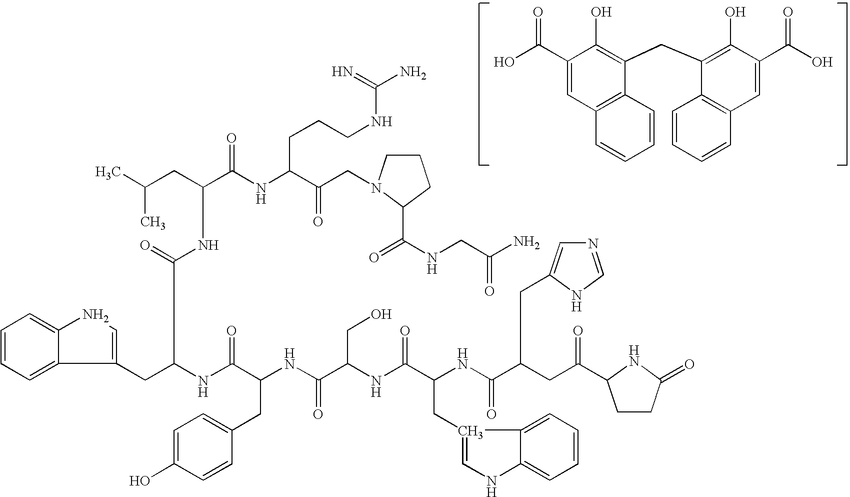
(triptorelin pamoate; sold as Trelstar® by Pharmacia Company, Kalamazoo, Mich.).

In an embodiment of the invention, an FPT inhibitor is provided in association with a progestational agent such as 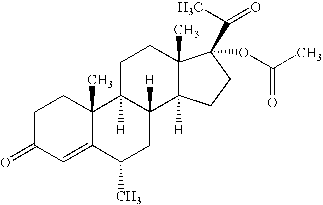
(medroxyprogesterone acetate, sold as Provera® by Pharmacia & Upjohn Co.; Kalamazoo, Mich.) 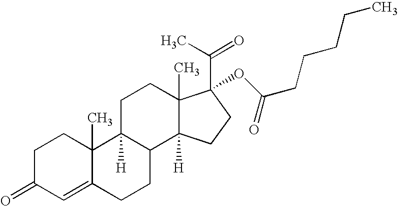
(hydroxyprogesterone caproate; 17-((1-Oxohexyl)oxy)pregn-4-ene-3,20-dione;) megestrol acetate or progestins.

In an embodiment of the invention, an FPT inhibitor is provided in association with selective estrogen receptor modulator (SERM) such as 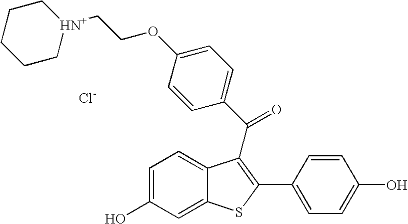
(raloxifene; sold as Evista® by Eli Lilly and Company; Indianapolis, Ind.).

In an embodiment of the invention, an FPT inhibitor is provided in association with an anti-androgen including, but not limited to: 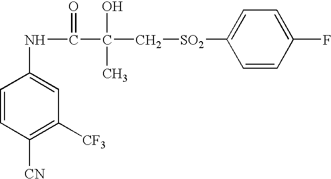
(bicalutamide; sold at CASODEX® by AstraZeneca Pharmaceuticals LP; Wilmington, Del.); 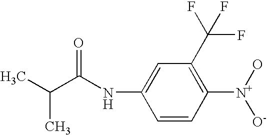
(flutamide; 2-methyl-N-[4-nitro-3 (trifluoromethyl) phenyl]propanamide; sold as Eulexin® by Schering Corporation; Kenilworth, N.J.); 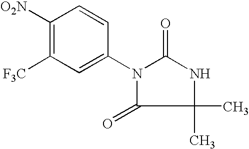
(nilutamide; sold as Nilandron® by Aventis Pharmaceuticals Inc.; Kansas City, Mo.) and 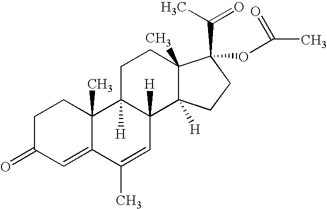
(Megestrol acetate; sold as Megace® by Bristol-Myers Squibb).

In an embodiment of the invention, an FPT inhibitor is provided in association with one or more inhibitors which antagonize the action of the EGF Receptor or HER2, including, but not limited to, CP-724714 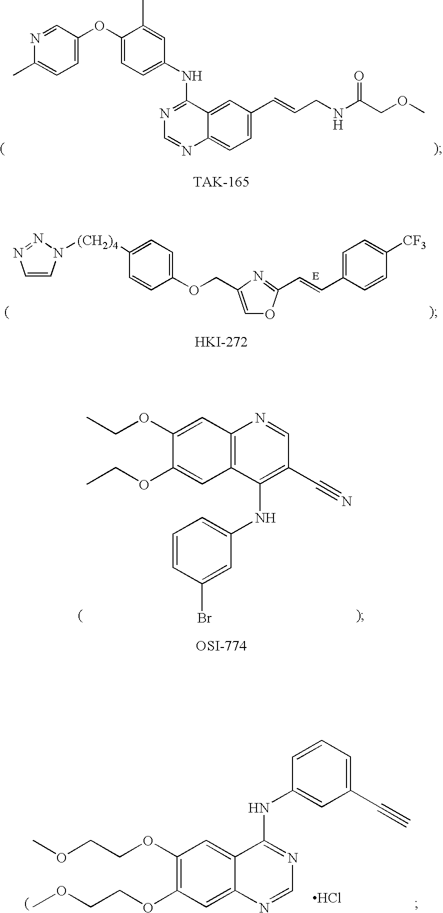
erlotinib, Hidalgo et al., J. Clin. Oncol. 19(13); 3267-3279 (2001)), Lapatanib 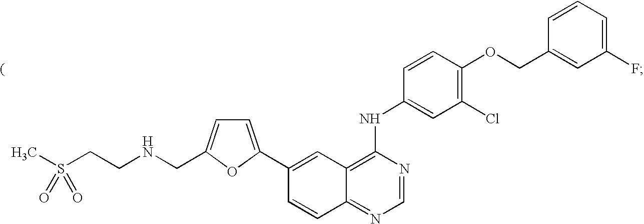
GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No WO99/35146), Canertinib (CI-1033; 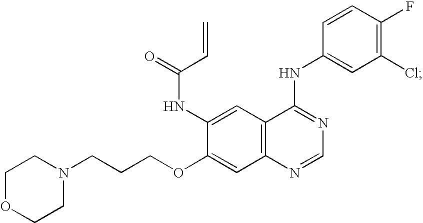
Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; Imclone; New York, N.Y.), EKB-569 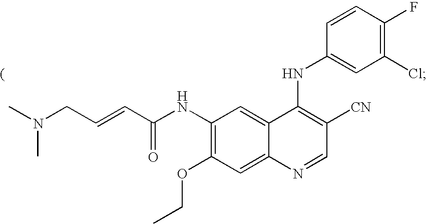
Wissner et al., J. Med. Chem. 46(1): 49-63 (2003)), PKI-166 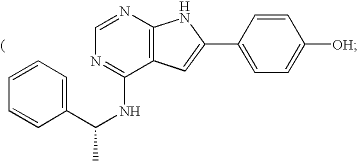
CGP-75166), GW-572016, any anti-EGFR antibody and any anti-HER2 antibody.

In an embodiment of the invention, an FPT inhibitor is provided in association with 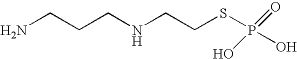
(Amifostine); 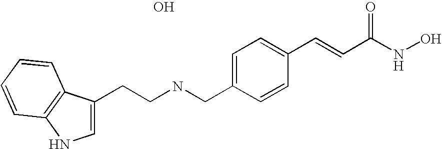
(NVP-LAQ824; Atadja et al., Cancer Research 64: 689-695 (2004)), 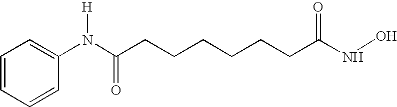
(suberoyl analide hydroxamic acid), 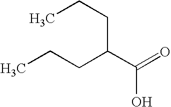
(Valproic acid; Michaelis et al., Mol. Pharmacol. 65:520-527 (2004)), 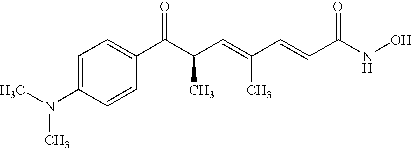
(trichostatin A), 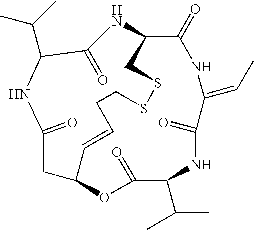
(FK-228; Furumai et al., Cancer Research 62: 4916-4921 (2002)), 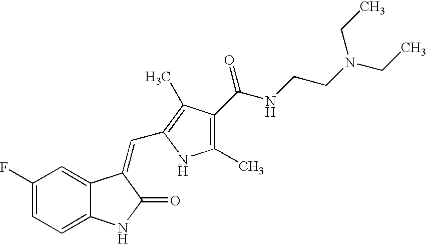
(SU11248; Mendel et al., Clin. Cancer Res. 9(1):327-37 (2003)), 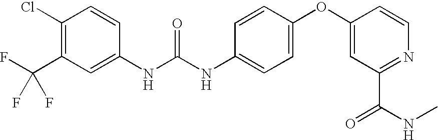
(BAY43-9006),

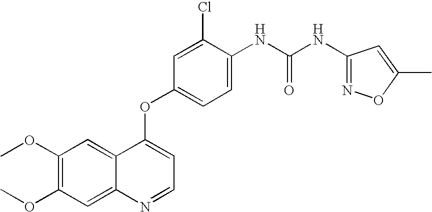

(KRN951), 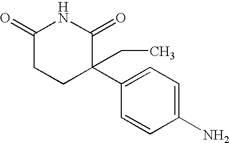
(Aminoglutethimide); 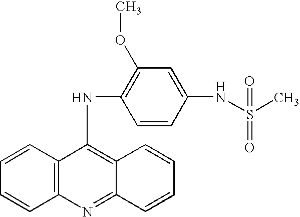
(Amsacrine); 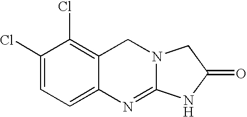
(Anagrelide); 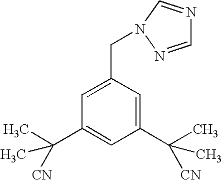
(Anastrozole; sold as Arimidex by AstraZeneca Pharmaceuticals LP, Wilmington, Del.); Asparaginase; Bacillus Calmette-Guerin (BCG) vaccine (Garrido et al., Cytobios. 90(360):47-65 (1997)); 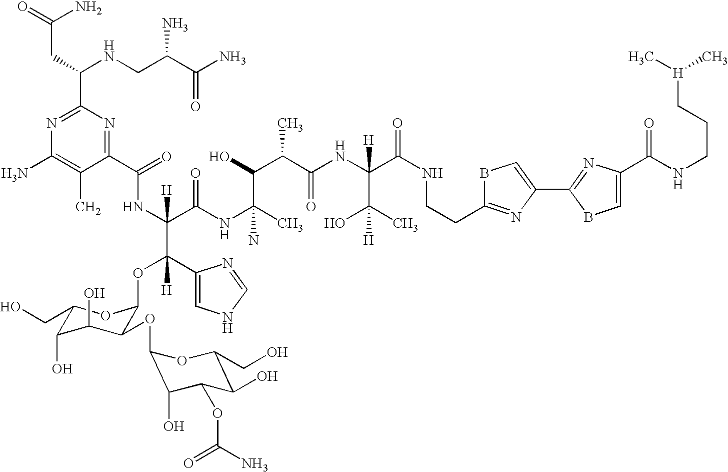
(Bleomycin); 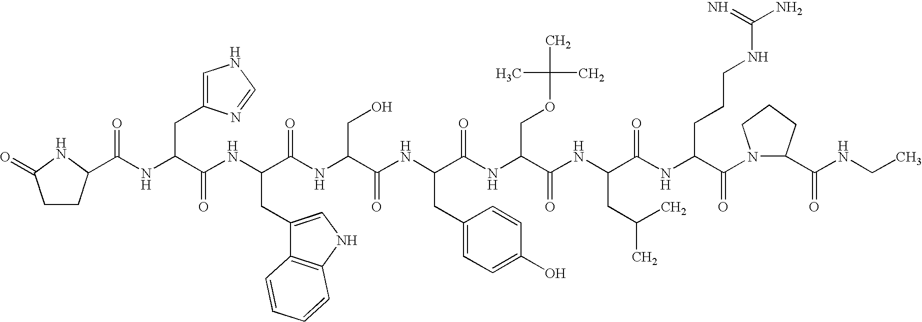
(Buserelin); 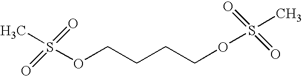
(Busulfan; 1,4-butanediol, diethanesulfonate, sold as Busulfex® by ESP Pharma, Inc.; Edison, N.J.); 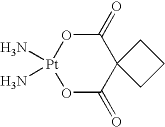
(Carboplatin; sold as Paraplatin® by Bristol-Myers Squibb; Princeton, N.J.); 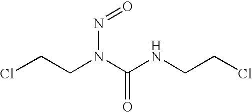
(Carmustine); 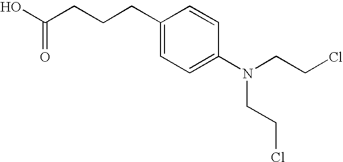
(Chlorambucil); 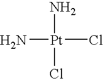
(Cisplatin); 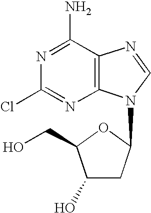
(Cladribine); 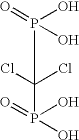
(Clodronate); 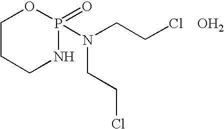
(Cyclophosphamide); 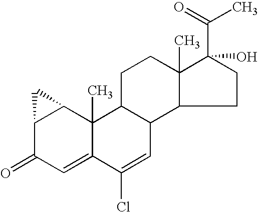
(Cyproterone); 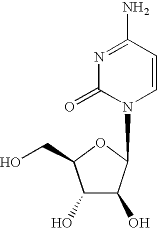
(Cytarabine); 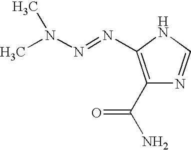
(Dacarbazine); 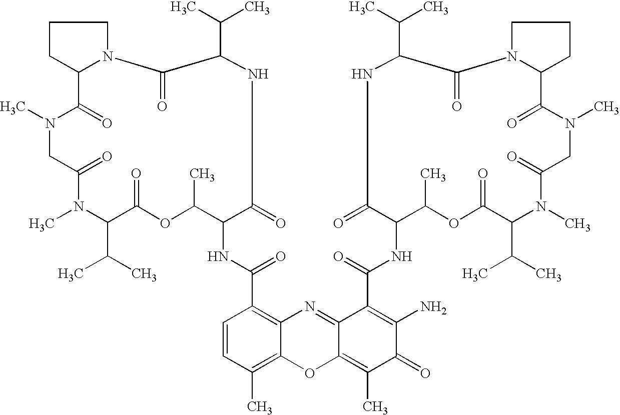
(Dactinomycin); 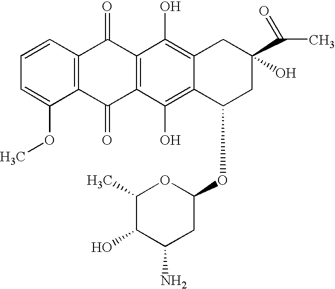
(Daunorubicin); 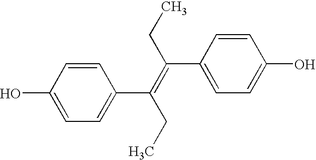
(Diethylstilbestrol); 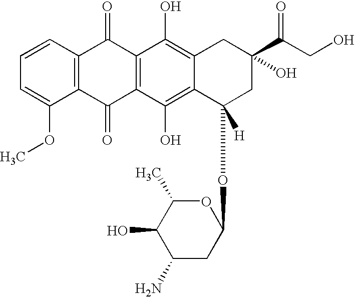
(Epirubicin); 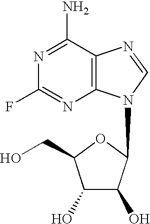
(Fludarabine); 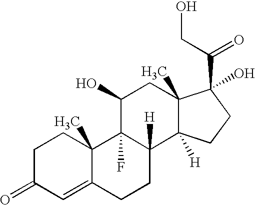
(Fludrocortisone); 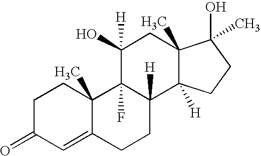
(Fluoxymesterone); 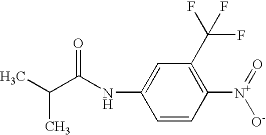
(Flutamide); 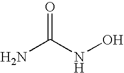
(Hydroxyurea); 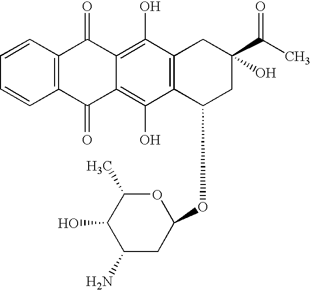
(Idarubicin); 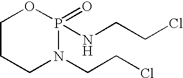
(Ifosfamide); 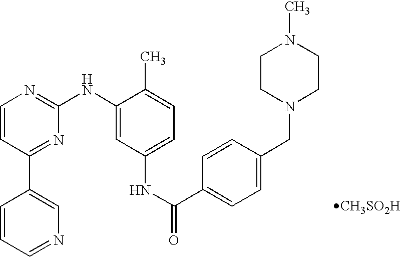
(Imatinib; sold as Gleevec® by Novartis Pharmaceuticals Corporation; East Hanover, N.J.); 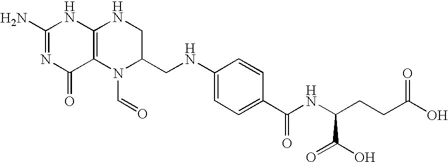
(Leucovorin); 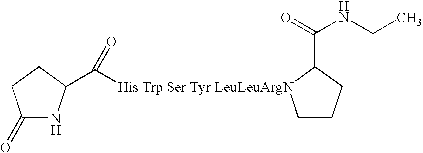
(Leuprolide); 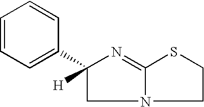
(Levamisole); 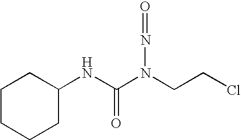
(Lomustine); 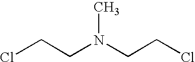
(Mechlorethamine); 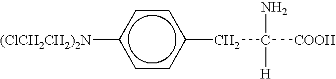
(Melphalan; sold as Alkeran® by Celgene Corporation; Warren, N.J.); 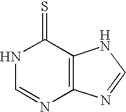
(Mercaptopurine); 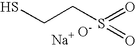
(Mesna); 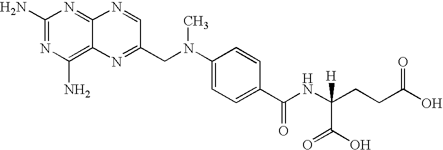
(Methotrexate); 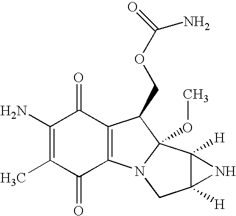
(Mitomycin); 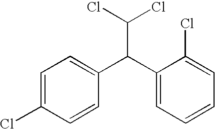
(Mitotane); 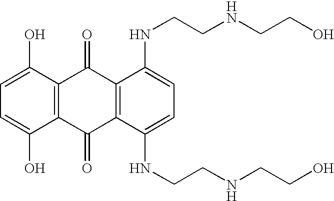
(Mitoxantrone); 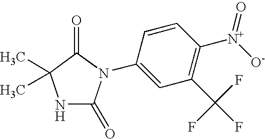
(Nilutamide); octreotide (L-Cysteinamide, D-phenylalanyl L-cysteinyl-L-phenylalanyl-D-tryptophyl-L-lysyl-L-threonyl-N-[2-hydroxy-1-(hydroxymethyl) propyl]-, cyclic (2_{—}7)-disulfide; [R R*,R*)];

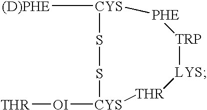

Katz et al., Clin Pharm. 8(4):255-73 (1989); sold as Sandostatin LAR® Depot; Novartis Pharm. Corp; E. Hanover, N.J.); oxaliplatin ( 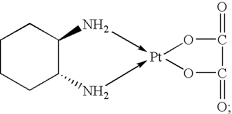
sold as Eloxatin™ by Sanofi-Synthelabo Inc.; New York, N.Y.); 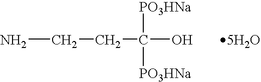
(Pamidronate; sold as Aredia® by Novartis Pharmaceuticals Corporation; East Hanover, N.J.); 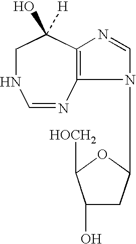
(Pentostatn; sold as Nipent® by Supergen; Dublin, Calif.); 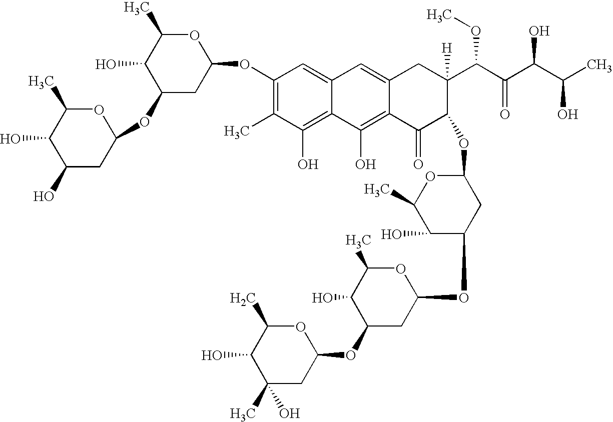
(Plicamycin); 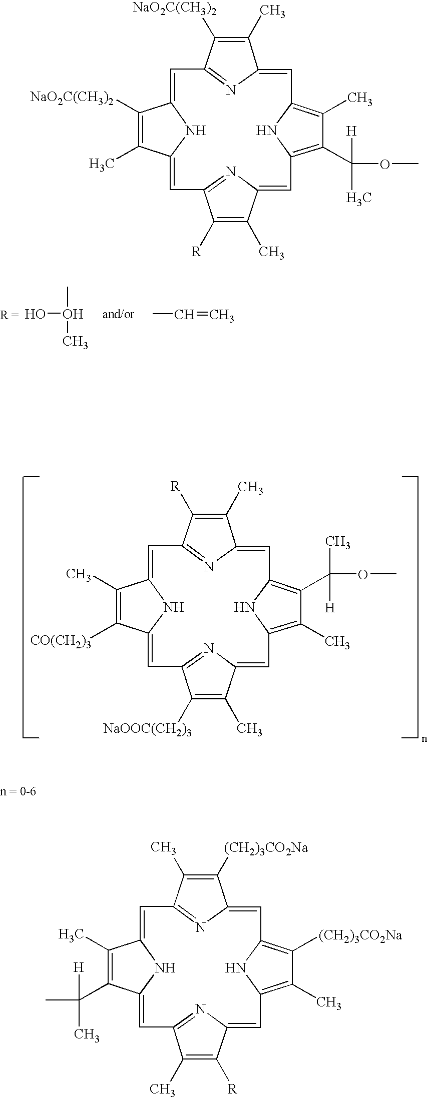
(Porfimer; sold as Photofrin® by Axcan Scandipharm Inc.; Birmingham, Ala.); 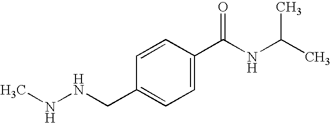
(Procarbazine); 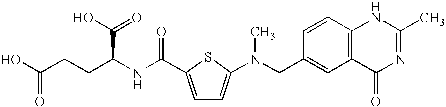
(Raltitrexed); Rituximab (sold as Rituxan® by Genentech, Inc.; South San Francisco, Calif.); 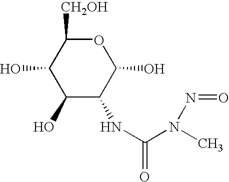
(Streptozocin); 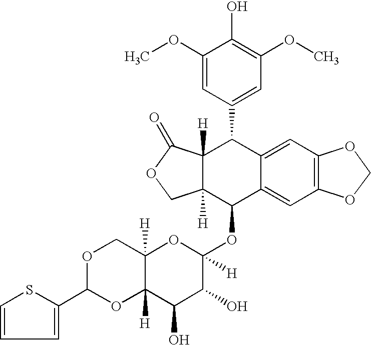
(Teniposide); 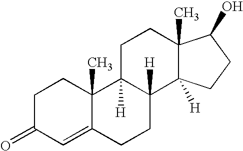
(Testosterone); 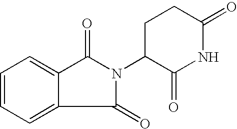
(Thalidomide); 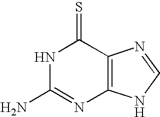
(Thioguanine), 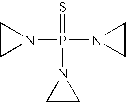
(Thiotepa); 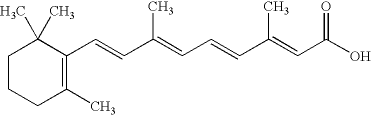
(Tretinoin); 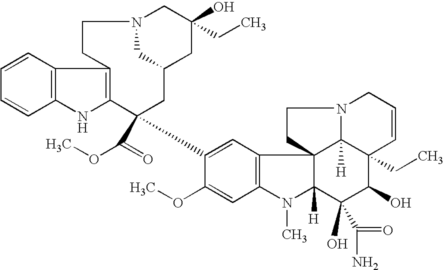
(Vindesine) or 13-cis-retinoic acid

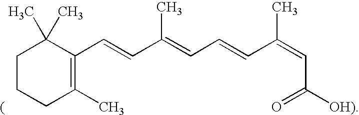

In an embodiment of the invention, an FPT inhibitor is provided in association with an IGF1R inhibitor such as for example BMS-577098 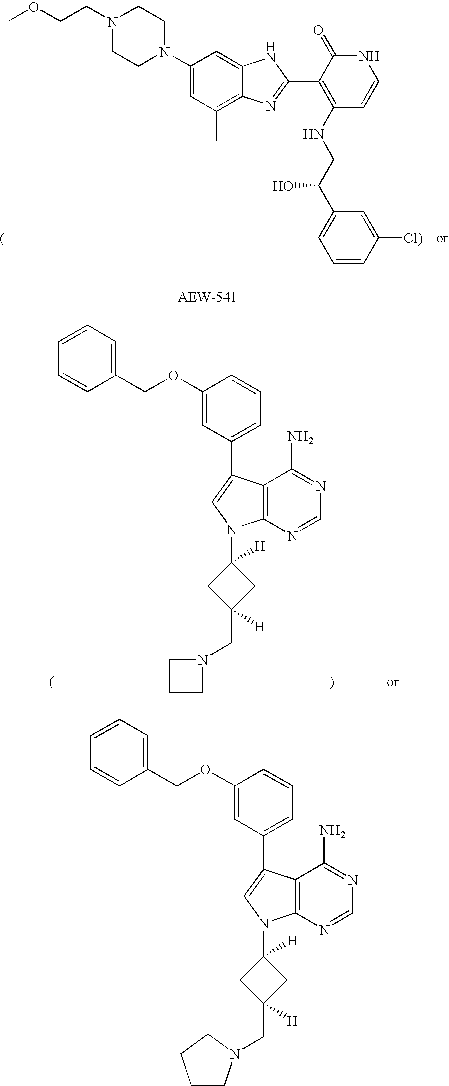
In an embodiment of the invention, an IGF1R inhibitor that is administered to a patient in a method according to the invention is an isolated anti-insulin-like growth factor-1 receptor (IGF1R) antibody comprising a mature 19D12/15H12 Light Chain-C, D, E or F and a mature 19D12/15H12 heavy chain-A or B. In an embodiment of the invention, an IGF1R inhibitor that is administered to a patient in a method according to the invention is an isolated antibody that specifically binds to IGF1R that comprises one or more complementarity determining regions (CDRs) of 19D12/15H12 Light Chain-C, 9, E or F and/or 19D12/15H12 heavy chain-A or B (e.g., all 3 light chain CDRs and all 3 heavy chain CDRs).

The amino acid and nucleotide sequences of antibody chains of the invention are shown below. Dotted, underscored type indicates the signal peptide. Solid underscored type indicates the CDRs. Plain type indicates the framework regions, Mature fragments lack the signal peptide.

Modified 19D12/15H12 Light Chain-C

(SEQ ID NO: 4)

[symbol]

[symbol]

GAA ATT GTG CTG ACT CAG AGC CCA GAC TCT
CTG TCT GTG ACT CCA GGC GAG AGA GTC ACC ATC ACC
TGC CGG GCC AGT CAG AGC ATT GGT AGT AGC TTA CAC
TGG TAC CAG CAG AAA CCA GGT CAG TCT CCA AAG CTT
CTC ATC AAG TAT GCA TCC CAG TCC CTC TCA GGG GTC
CCC TCG ACG TTC AGT GGC AGT GGA TCT GGG ACA GAT
TTC ACC CTC ACC ATC AGT AGC CTC GAG GCT GAA GAT
GCT GCA GCG TAT TAC TGT CAT CAG AGT AGT CGT TTA
CCT CAC ACT TTC GGC CAA GGG ACC AAG GTG GAG ATC
AAA CGT ACG

(SEQ ID NO: 5)

[symbol]

[symbol]

E I VI L T Q S P D S L S V T P G E R V T I T C
R A S Q S I G S S L H W Y Q Q K P G Q S P K L L I
K Y A S Q S L S G V P S R F S G S GS G T D F T L T
I S S L E A E D A A A Y Y C H Q S S R L P H T F G
Q G T K V E I K R T
Modified 19D12/15H12 Light Chain-D

(SEQ ID NO: 6)

[symbol]

[symbol]

GAA ATT GTG CTG ACT CAG AGC CCA GAC TCT
CTG TCT GTG ACT CCA GGC GAG AGA GTC ACC ATC ACC
TGC CGG GCC AGT CAG AGC ATT GGT AGT AGC TTA CAC
TGG TAC CAG CAG AAA CCA GGT CAG TCT CCA AAG CTT
CTC ATC AAG TAT GCA TCC CAG TCC CTC TCA GGG GTC
CCC TCG AGG TTC AGT GGC AGT GGA TCT GGG ACA GAT
TTC ACC CTC ACC ATC AGT AGC CTC GAG GCT GAA GAT
TTC GCA GTG TAT TAC TGT CAT CAG AGT AGT CGT TTA
CCT CAC ACT TTC GGC CAA GGG ACC AAG GTG GAG ATC
AAA CGT ACG

(SEQ ID NO: 7)

[symbol]

[symbol]

E I V L T Q S P D S L S V T P G E R V T I T I C
R A S Q S I G S S L H W Y Q Q K P G Q S P K L L I
K Y A S Q S L S G V P S R F S G S G S G T D F T L
T I S S L E A E D F A V Y Y C H Q S S R L P H T F
G Q G T K V E I K R T
Modified 19D12/15H12 Light Chain-E

(SEQ ID NO: 8)

[symbol]

[symbol]

GAA ATT GTG CTG ACT CAG AGC CCA GGT ACC CTG
TCT GTG TCT CCA GGC GAG AGA GCC ACC CTC TCC TGC
CGG GCC AGT CAG AGC ATT GGT AGT AGC TTA CAC TGG
TAC CAG CAG AAA CCA GGT CAG GCT CCA AGG CTT CTC
ATC AAG TAT GCA TCC CAG TCC CTC TCA GGG ATC CCC
GAT AGG TTC AGT GGC AGT GGA TCT GGG ACA GAT TTC
ACC CTC ACC ATC AGT AGA CTG GAG CCT GAA GAT GCT
GCA GCG TAT TAC TGT CAT CAG AGT AGT CGT TTA CCT
CAC ACT TTC GGC CAA GGG ACC AAG GTG GAG ATC AAA
CGT ACA

(SEQ ID NO: 9)

[symbol]

[symbol]

E I V L T Q S P G T L S V S P G E R A T L S C
R A S Q S I G S S L H W Y Q Q K P G Q A P R L L I
K Y A S Q S L S G I P D R F S G S G S G T D F T L
T I S S R L E P E D A A A Y Y C H Q S S R L P H T
F G Q G T K V E I K R T
Modified 19D12/15H12 Light Chain-F

(SEQ ID NO: 10)

[symbol]

[symbol]

GAA ATT GTG CTG ACT CAG AGC CCA GGT ACC
CTG TCT GTG TCT CCA GGC GAG AGA GCC ACC CTC TCC
TGC CGG GCC AGT CAG AGC ATT GGT AGT AGC TTA CAC
TGG TAC CAG CAG AAA CCA GGT CAG GCT CCA AGG CTT
CTC ATC AAG TAT GCA TCC CAG TCC CTC TCA GGG ATC
CCC GAT AGG TTC AGT GGC AGT GGA TCT GGG ACA GAT
TTC ACC CTC ACC ATC AGT AGA CTG GAG CCT GAA GAT
TTC GCA GTG TAT TAC TGT CAT CAG AGT AGT CGT TTA
CCT CAC ACT TTC GGC CAA GGG ACC AAG GTG GAG ATC
CGT ACA

(SEQ ID NO: 11)

[symbol]

[symbol]

E IV L T Q S P G T L S V S P G E R A T L S C
R A S Q S I G S S L H W Y Q Q K P G Q A P R L L I
K Y A S Q S L S G I P D R F S G S G S G T D F T L
T I S R L E P E D F A V Y Y C H Q S S R L P H T F
G Q G T K V E I K R T
Modified 19D12/15H12 heavy chain-A

(SEQ ID NO: 12)

[symbol]

[symbol]

[symbol]

GAG GTT CAG CTG GTG CAG TCT GGG GGA GGC
TTG GTA AAG CCT GGG GGG TCC CTG AGA CTC TCC TGT
GCA GCC TCT GGA TTC ACC TTC AGT AGC TTT GCT ATG
CAC TGG GTT CGC CAG GCT CCA GGA AAA GGT CTG GAG
TGG ATA TCA GTT ATT GAT ACT CGT GGT GCC ACA TAC
TAT GCA GAC TCC GTG AAG GGC CGA TTC ACC ATC TCC
AGA GAC AAT GCC AAG AAC TCC TTG TAT CTT CAA ATG
AAC AGC CTG AGA GCC GAG GAC ACT GCT GTG TAT TAC
TGT GCA AGA CTG GGG AAC TTC TAC TAC GGT ATG GAC
GTC TGG GGC CAA GGG ACC ACG GTC ACC GTC TCC TCA

(SEQ ID NO: 13)

[symbol]

[symbol]

[symbol]

Glu Val Gln Leu Val Gln Ser Gly Gly Gly
Leu Val Lys Pro Gly Gly Ser Leu Arg Leu Ser Cys
Ala Ala Ser Gly Phe Thr Phe Ser Ser Phe Ala Met
His Trp Val Arg Gln ALa Pro GLy Lys Gly Leu Glu
Trp Ile Ser Val Ile Asp Thr Arg Gly Ala Thr Tyr
Tyr Ala Asp Ser Val Lys Gly Arg Phe Thr Ile Ser
Arg Asp Asn Ala Lys Asn Ser Leu Tyr Leu Gln Met
Asn Ser Leu Arg Ala Glu Asp Thr Ala Val Tyr Tyr
Cys Ala Arg Leu Gly Asn Phe Tyr Tyr Gly Met Asp
Val Trp GLy GLn Gly Thr Thr Val Thr Val Ser Ser
Modified 19D12/15H12 heavy chain-B

(SEQ ID NO: 14)

[symbol]

[symbol]

GAG GTT CAG CTG GTG CAG TCT GGG GGA GGC
TTG GTA CAG CCC GGG GGG TCC CTG AGA CTC TCC TGT
GCA GCC TCT GGA TTC ACC TTC AGT AGC TTT GCT ATG
CAC TGG GTT CTC CAG GCT CCA GGA AAA GGT CTG GAG
TGG ATA TCA GTT ATT GAT ACT CGT GGT GCC ACA TAC
TAT GCA GAC TCC GTG AAG GGC CGA TTC ACC ATC TCC
AGA GAC AAT GCC AAG AAC TCC TTG TAT CTT CAA ATG
AAC AGC CTG AGA GCC GAG GAC ACT GCT GTG TAT TAC
TGT GCA AGA CTG GGG AAC TTC TAC TAC GGT ATG GAC
GTC TGG GGC CAA GGG ACC ACG GTC ACC GTC TCC TCA

(SEQ ID NO: 15)

[symbol]

[symbol]

Glu Val Gln Leu Val Gln Ser Gly Gly Gly
Leu Val Gln Pro Gly Gly Ser Leu Arg Leu Ser Cys
Ala Ala Ser Gly Phe Thr Phe Ser Ser Phe Ala Met
His Trp Val Arg Gln Ala Pro Gly Lys Gly Leu Glu
Trp Ile Ser Val Ile Asp Thr Arg Gly Ala Thr Tyr
Tyr Ala Asp Ser Val Lys Gly Arg Phe Thr Ile Ser
Arg Asp Asn ALa Lys Asn Ser Leu Tyr Leu Gln Met
Asn Ser Leu Arg Ala Glu Asp Thr Ala Val Tyr Tyr
Cys ALa Arg Leu Gly Asn Phe Tyr Tyr Gly Met Asp
Val Trp Gly Gln GLy Thr Thr Val Thr Val Ser Ser

In an embodiment, an antibody that binds “specifically” to human IGF1R binds with a Kd of about 10⁻⁸ M or 10⁻⁷ M or a lower number; or, in an embodiment of the invention, with a Kd of about 1.28×10⁻¹⁰ M or a lower number by Biacore measurement or with a Kd of about 2.05×10⁻¹² or a lower number by KinExA measurement. In another embodiment, an antibody that binds “specifically” to human IGF1R binds exclusively to human IGF1R and to no other protein.

In an embodiment of the invention, an FPT inhibitor is provided in association with one or more of any of: phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-33359, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), rapamycin, CCI-779 (Sehgal et al., Med. Res. Rev., 14:1-22 (1994); Elit, Curr. Opin. Investig. Drugs 3(8):1249-53 (2002)), LY294002, LY292223, LY292696, LY293684, LY293646 (Vlahos et al., J. Biol. Chem. 269(7): 5241-5248 (1994)), wortmannin, BAY43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)).

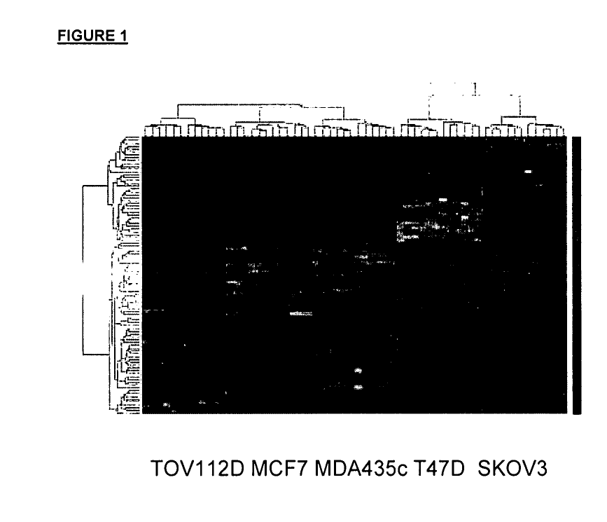

In an embodiment of the invention, an FPT inhibitor is provided in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR, in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylamino)-8-methyl-8H-pyrido(2,3-d)pyrimidin-7-one.

In an embodiment of the invention, an FPT inhibitor is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n-1 pegylated or unpegylated interferon alfa n-3 and pegylated, unpegylated consensus interferon or albumin-interferon-alpha.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3 a mixture of natural alpha interferons.

Interferon alfa-2a is sold as ROFERON-A® by Hoffmann-La Roche (Nutley, N.J.).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “112,000 molecular weight polyethylene glycol conjugated interferon alpha” and “PEG 12000-IFN alpha” as used herein include conjugates such as are prepared according to the methods of International Application No. WO 95/13090 and containing urethane linkages between the interferon alpha-2a or -2b amino groups and polyethylene glycol having an average molecular weight of 12000. The pegylated interferon alpha, PEG 12000-IFN-alpha-2b is available from Schering-Plough Research Institute, Kenilworth, N.J.

The preferred PEG 12000-interferon alpha-2b can be prepared by attaching a PEG polymer to the epsilon amino group of a lysine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J.).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917,888, European Patent Application No. 0 236 987 or 0 593 868 or International Publication No. WO 95/13090.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes compositions comprising an FPT inhibitor in association with one or more other anti-cancer chemotherapeutic agents (e.g., as described herein) and optionally (i.e., with or without) in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co.; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine™), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kyril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering an FPT inhibitor optionally in association with one or more other chemotherapeutic agents (e.g., as described herein) and optionally in association with one or more antiemetics.

### Pharmaceutical Compositions, Dosage and Administration

The present invention comprises methods for treating or preventing any medical condition mediated by farnesylation with a farnesyl protein transferase (e.g., any hyperproliferative disease such as cancer).

Within the scope of the invention are methods wherein a patient is assessed as a possible candidate for treatment with a farnesyl protein transferase inhibitor. Such an assessment can take the form of obtaining a cell from a tumor in the patient and determining the expression level of biomarkers (as set forth herein) in the cell. If one or more of the biomarkers of table 1 (e.g., PRL2, claudin-1, mucin-1, LTB4DH and endothelin-1), in the tumor cell, are expressed at a lower level than that of a cell line known to be resistant to the inhibitor, then the tumor cell is likely to be sensitive to the inhibitor. Similarly, if one or more of the biomarkers of table 2 (e.g., PDGFRL), in the tumor cell, are expressed at a higher level than that of a cell line known to be resistant to the inhibitor, then the tumor cell is likely to be sensitive to the inhibitor. If the tumor cell is determined to be sensitive, then the patient is, in turn, determined to be a candidate for treatment with the inhibitor. Ideally, though, by no means necessarily, all biomarkers in table 1 will be underexpressed in the tumor cell and all biomarkers in table 2 will be overexpressed in the tumor cell relative to a resistant cell line.

The present invention includes methods wherein a tumor cell is determined to be sensitive to a farnesyl protein transferase inhibitor if it has the expression profile described below in tables 1 and 2 (i.e., all genes therein or one or more genes). Specifically, wherein the tumor cell tested underexpresses or overexpresses all of the genes set forth in tables 1 and 2, respectively, as compared to a farnesyl protein transferase inhibitor resistant cell (e.g., T47D or SKOV3 or any other cell exhibiting an IC50 of ≧1000 nM to a farnesyl protein transferase inhibitor such as lonafarnib). In an embodiment of the invention, only genes in table 1 or 2 for which there is an accession number indicated are considered when evaluating the sensitivity of a given cell to an FTI. In an embodiment of the invention, the tumor cell is determined to be sensitive to a farnesyl protein transferase inhibitor if it underexpresses or overexpresses any genes to any degree whatsoever or at least to the degree set forth in the tables.

In an embodiment of the invention, a cell is considered to be FTI sensitive if it:
(i) expresses less e.g. ≧about 16 times (e.g., about 8, 9, 10, 12, 13, 14, 15, 16, 17, 18, 19, 20 or 25 times less) claudin-1 (e.g., mRNA) than an FTI (e.g., lonafarnib) resistant cell line (e.g., T47D); and/or
(ii) expresses less e.g., ≧about 13 times (e.g., about 6, 7, 8, 9, 10, 12, 13, 14, 15, 16, 17, 18, 19, 20 or 22 times less) mucin-1 (e.g., mRNA) than an FTI (e.g., lonafarnib) resistant cell line (e.g., T47D); and/or
(iii) expresses less e.g., ≧about 4 times (e.g., about 2, 3, 4, 5, 6, 7, 8, 9 or 10 times less) PRL2 (e.g., mRNA) than an FTI (e.g., lonafarnib) resistant cell line (e.g., T47D); and/or
(iv) expresses less e.g., ≧about 4 times (e.g., about 2, 3, 4, 5, 6, 7, 8, 9 or 10 times less) LTB4DH (e.g., mRNA) than an FTI (e.g., lonafarnib) resistant cell line (e.g., T47D); and/or
(v) expresses less e.g., ≧about 3 times (e.g., about 2, 3, 4, 5, 6, 7, 8, 9 or 10 times less) endothelin-1 (e.g., mRNA) than an FT e.g., lonafarnib) resistant cell line (e.g., T47D); and/or
(vi) expresses more e.g., ≧about 99 times (e.g., about 45, 50, 60, 65, 70, 75, 100, 110, 115, 120, 130 or 200 times more) PDGFRL (e.g., mRNA) than an FTI (e.g., lonafarnib) resistant cell line (e.g., T47D)
   (including any possible combination thereof).
   A cell comprising any one of the foregoing characteristics ((i)-(vi)), all of the characteristics or any combination thereof (e.g., (i), (ii) and (vi) or (i), (iii), (iv), (v) and (vi)) is considered an FTI (e.g., lonafarnib) sensitive cell.

The cancer need not, in all cases, be determined, in the methods of the present invention, as absolutely FTI resistant or sensitive. The present invention includes embodiments wherein the relative level of FTI sensitivity or resistance, as compared to that of other cell lines, is assessed. For example, in one embodiment of the invention, a colorectal tumor's cells assessed for PRL2 expression levels might be determined to be only moderately FTI sensitive or highly FTI resistant but not completely FTI resistant. This judgment can be reached, for example, by comparing the level of PRL2 expression to that of other cell lines which are commonly known to be FTI resistant (e.g., as discussed herein). As discussed above, based on the assessment of a cancer's relative FTI sensitivity or resistance, a clinician or doctor of ordinary skill in the art may make a reasoned decision, based on, e.g., the particular needs of the patient involved, other regimens the patient is receiving, and the exigencies of the particular situation as to whether to undertake a treatment regimen with a given FTI.

If a tumor is identified using the criteria set forth herein to comprise FTI sensitive cells, the patient with the cells can be identified as a candidate for FTI therapy, selected and treated accordingly.

The present invention also includes embodiments wherein a patient's blood levels of endothelin-1 are assessed. If the patient's endothelin-1 blood levels are above the range normally observed in a patient, then any cancer from which the patient is suffering can be determined to be FTI (e.g., lonafarnib) resistant. For example, in an embodiment of the invention, normal blood levels of endothelin-1 are about 0.2 to about 5 pg/ml.

In an embodiment of the invention, the cancer is one or more of lung cancer (e.g., lung adenocarcinoma and non small cell lung cancer), pancreatic cancer (e.g., pancreatic carcinoma such as, for example, exocrine pancreatic carcinoma), colon cancer (e.g., colorectal carcinomas, such as, for example, colon adenocarcinoma and colon adenoma), myeloid Leukemia (for example, acute myelogenous leukemia (AML), CML, and CMML), thyroid follicular cancer, myelodysplastic syndrome (MDS), bladder carcinoma, epidermal carcinoma, melanoma, breast cancer, prostate cancer, head and neck cancer (e.g., squamous cell cancer of the head and neck), ovarian cancer, brain cancer (e.g., gliomas), cancer of mesenchymal origin (e.g., fibrosarcomas and rhabdomyosarcomas), sarcoma, tetracarcinoma, neuroblastoma, kidney carcinoma, hepatoma, non-Hodgkin's lymphoma, multiple myeloma and anaplastic thyroid carcinoma.

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker. See also U.S. Pat. No. 6,632,455; and European patent no. 1039908.

Inert, pharmaceutically acceptable carriers used for preparing pharmaceutical compositions of FPT inhibitors described herein can be solid or liquid. Solid preparations include powders, tablets, dispersible granules, capsules, cachets and suppositories. The powders and tablets may, in an embodiment of the invention, comprise from about 5 to about 70% active ingredient. Solid carriers are known in the art, e.g., magnesium carbonate, magnesium stearate, talc, sugar, and/or lactose. Tablets, powders, cachets and capsules can. In an embodiment of the invention, be used as solid dosage forms suitable for oral administration.

In an embodiment of the invention, for preparing suppositories, a low melting wax such as a mixture of fatty acid glycerides or cocoa butter is first melted, and the active ingredient is dispersed homogeneously therein as by stirring. The molten homogeneous mixture is then poured into conveniently sized molds, allowed to cool and thereby solidify.

Liquid preparations include, in an embodiment of the invention, solutions, suspensions and emulsions. As an example may be mentioned water or water-propylene glycol solutions for parenteral injection. Liquid preparations may also include, in an embodiment of the invention, solutions for intranasal administration.

Aerosol preparations suitable for inhalation may include, in an embodiment of the invention, solutions and solids in powder form, which may be in combination with a pharmaceutically acceptable carrier, such as an inert compressed gas.

Also included in an embodiment of the invention are solid preparations which are intended for conversion, shortly before use, to liquid preparations for either oral or parenteral administration. Such liquid forms include, in an embodiment of the invention, solutions, suspensions and emulsions.

The FPT inhibitors described herein may also be deliverable, in an embodiment of the invention, transdermally. The transdermal compositions can take the form of creams, lotions, aerosols and/or emulsions and can be included in a transdermal patch of the matrix or reservoir type as are conventional in the art for this purpose.

In an embodiment of the invention, the FPT inhibitors are administered orally. In an embodiment of the invention, the pharmaceutical preparation is in unit dosage form. In such a form, the preparation is subdivided into unit doses containing appropriate quantities of the active component, e.g., an effective amount to achieve the desired purpose.

In an embodiment of the invention, the quantity of active compound in a unit dose of preparation is varied or adjusted from about 0.5 mg to 1000 mg, preferably from about 1 mg to 300 mg, more preferably 5 mg to 200 mg, according to the particular application.

In an embodiment of the invention, a therapeutically effective dosage or amount of any chemotherapeutic agent (e.g., as set forth herein) is, whenever possible, as set forth in the Physicians' Desk Reference 2003 (Thomson Healthcare, 57th edition (Nov. 1, 2002)) which is herein incorporated by reference or in the scientific literature.

The actual dosage employed may be varied depending upon the requirements of the patient and the severity of the condition being treated. Determination of the proper dosage for a particular situation is within the skill of the art. In an embodiment of the invention, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

A physician or clinician may use any of several methods known in the art to measure the effectiveness of a particular dosage scheme of a chemotherapeutic therapeutic agent. For example, tumor size can be determined in a non-invasive route, such as by X-ray, positron emission tomography (PET) scan, computed tomography (CT) scan or magnetic resonance imaging (MRI).

In an embodiment of the invention, a therapeutically effective amount of an FPT inhibitor (e.g., lonafarnib) is about 200 mg BID (twice daily).

In a combination therapy embodiment of the present invention, a low dosage regimen of the FPT inhibitors is, e.g., oral administration of an amount in the range of from 1.4 to 400 mg/day, e.g., 1.4 to 350 mg/day, or 3.5 to 70 mg/day, e.g., with a B.I.D. dosing schedule. A particularly low dosage range can, in an embodiment of the invention, be 1.4 to 70 mg/day.

In an embodiment of the invention, a therapeutically effective dosage of lonafarnib and a taxane, such as paclitaxel, when co-administered, is as follows: lonafarnib (e.g., capsules taken orally) twice daily with food at 50 mg, 75 mg, 100 mg or 200 mg with the paclitaxel (e.g., administered intravenously) every 3 weeks at 135 mg/m² or 175 mg/m² over 3 h (see e.g., Khuri et al., Clinical Cancer Research 10: 2968-2976 (2004)).

In an embodiment of the invention, a therapeutically effective dosage of lonafarnib and docetaxel, temozolomide or anastrazole is about 200 mg BID lonafarnib and the approved dosage of docetaxel, temozolomide or anastrazole. In an embodiment of the invention, the docetaxel regimen is for treatment of prostate cancer.

In an embodiment, a therapeutically effective dosage of any anti-IGF1R antibody (e.g., 19D12/15H12 LCF/HCA), which may be administered in association with an FPT inhibitor is in the range of about 0-3 mg/kg (body weight) to about 20 mg/kg (e.g., 0.3 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg) per day (e.g., 1 time, 2 times or 3 times per week).

In an embodiment of the invention, any antineoplastic agent used with an FPT inhibitor is administered in its normally prescribed dosages during the treatment cycle (i.e., the antineoplastic agents are administered according to the standard of practice for the administration of these drugs).

In an embodiment of the invention, lonafarnib is administered to treat advanced urothelial tract cancer at 150 mg in the morning and 100 mg in the evening along with gemcitabine at 1000 mg/m² on day 1, 8 and 15 per 28-day cycle (Theodore et al. Eur. J. Cancer (2005) 41(8):1150-7).

In an embodiment of the invention, lonafarnib is administered to treat solid cancers (e.g., non-small cell lung cancer) p.o., twice daily (b.i.d.) on continuously scheduled doses of 100 mg or 125 mg or 150 mg in combination with intravenous paclitaxel at doses of 135 mg/m² or 175 mg/m² administered over 3 hours on day 8 of every 21-day cycle (Khuri et al., Clin. Cancer Res. (2004) 10(9):2968-76).

In an embodiment of the invention, lonafarnib is administered to treat chronic myelogenous leukemia (CML) at 200 mg orally twice daily (Borthakur et al., Cancer (2006)106(2):346-52).

In an embodiment of the invention, lonafarnib is administered to treat taxane-refractory/resistant non-small cell lung carcinoma at 100 mg orally twice per day beginning on Day 1 and paclitaxel 175 mg/m² intravenously over 3 hours on Day 8 of each 21-day cycle (Kim et al., Cancer (2005) 104(3):561-9).

### Detection of Biomarkers

The determination of the expression level of a biomarker of the invention (as set forth herein) in a cancerous cell (e.g., in a tumor cell) can be performed using any of the many methods known in the art. In an embodiment of the invention, expression is determined by RT-PCR (real time PCR), Northern blot, Western blot, ELISA (enzyme linked immunosorbent assay), RIA (radioimmunoassay), gene chip analysis of RNA expression, immunohistochemistry or immunofluorescence. Embodiments of the invention include methods wherein biomarker RNA expression (transcription) is determined as well as methods wherein protein expression is determined. For example a laboratory technician can evaluate tumor biopsy samples from a potential candidate for farnesyl protein transferase inhibitor therapy using any of the foregoing analytical techniques (and others). Tumor biopsy techniques are well within the scope of ordinary knowledge of any surgeon (veterinary or human) or clinician.

In an embodiment of the invention, a tumor tissue biopsy is obtained and the cells in the tumor tissue are assayed for determination of biomarker expression. For northern blot or RT-PCR analysis, RNA should be isolated from the tumor tissue sample using RNAse free techniques. Such techniques are commonly known in the art.

Northern blot analysis of biomarker transcription in a tumor cell sample is, in an embodiment of the invention, performed. Northern analysis is a standard method for detection and quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. If a radiolabeled probe was used, the blot can be wrapped in plastic wrap to keep it from drying out and then immediately exposed to film for autoradiography. If a nonisotopic probe was used, the blot must generally be treated with nonisotopic detection reagents prior to film exposure. The relative levels of expression of the genes being assayed can be quantified using, for example, densitometry.

Biomarker expression is determined, in an embodiment of the invention, using RT-PCR. RT-PCR allows detection of the progress of a PCR amplification of a target gene in real time. Design of the primers and probes required to detect expression of a biomarker of the invention is within the skill of a practitioner of ordinary skill in the art. RT-PCR can be used to determine the level of RNA encoding a biomarker of the invention in a tumor tissue sample. In an embodiment of the invention, RNA from the tissue sample is isolated, under RNAse free conditions, then converted to DNA by treatment with reverse transcriptase. Methods for reverse transcriptase conversion of RNA to DNA are well known in the art.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5, end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

Expression of proteins encoded by biomarkers can also be detected in a tissue of a patient's tumor by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are “probed” using antibodies specific to the protein. Antibodies that recognize a protein in a band on the membrane will bind to it. The bound antibodies are then bound by a secondary anti-antibody antibody which is conjugated with a detectable label (e.g., biotin, horseradish peroxidase or alkaline phosphatase). Detection of the secondary label signal indicates the presence of the protein.

In an embodiment of the invention, expression of a protein encoded by a biomarker is detected by enzyme-linked immunosorbent assay (ELISA). In an embodiment of the invention, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In an embodiment of the invention, the expression of a biomarker is evaluated by use of a gene chip or microarray. Such techniques are within ordinary skill held in the art. An example of such a procedure is set forth below in the Examples section.

A sample from a tumor which can be assayed for the presence of a biomarker can come, for example, from a biopsy sample. Collection of a biopsy is well within the skill held by the ordinary doctor or clinician.

### EXAMPLES

The present invention is intended to exemplify the present invention and not to be a limitation thereof. Any method or composition disclosed below falls within the scope of the present invention.

### Example 1

### Identification of Biomarkers

In this example, biomarkers which are upregulated or downregulated in lonafarnib sensitive cell lines, relative to that of resistant cell lines T47D and SKOV3 were identified.

RNA Isolation

Cells were grown in 10 cm plates in triplicate and treated with DMSO or lonafarnib for 24 or 72 hours. The cells were then pelleted and snap frozen in liquid nitrogen and stored at −80° C. RNA was isolated using the Trizol reagent, following the manufacturer's instructions, and further purified the RNA by passing it over an RNAeasy column from Qiagen. RNA quantity and quality was assessed by measuring OD260/280 ratios and by gel electrophoresis.

Microarrays

Approximately 5 ug of total RNA was used for first and second strand cDNA synthesis. After purifications the cDNAs were in vitro transcribed to cRNAs. The biotinylated cRNAs were then fragmented and hybridized to Affymetrix Human U133 plus 2.0 arrays, according to the manufacturer's instructions (Affymetrix, Inc.; Santa Clara, Calif.).

Statistical Analysis

Data was analyzed using ArrayAnalyzer, and S+ based analysis tools Briefly, the data was scaled to a target value of 150 using MAS 5.0. The data was log 2 transformed and filtered by removing genes whose expression was called absent in all experiments and/or whose expression level was based on less than 7 of the 11 probe set pairs. In addition, control genes (AFFX prefix) were also removed before subsequent analysis. The data was then normalized on a per chip basis to the median IQR. This resulted in the removal of 16,066 genes from the dataset, leaving 38,568 genes to work with. Pairwise t-tests were then used to make the following comparisons—MCF7 v.s T47D (resistant cell lines, MDA435 vs. T47D (resistant cell line) and SKOV (resistant cell line) vs. TOV122. MCF7 and MDA435 are breast cancer cell lines which are commonly known in the art. A p value of 0.01 was used as well as the BH adjustment to control for false discovery. Overlap of these gene lists were determined using Venn diagrams. The overlap of these three gene lists resulted in 264 genes in common. 97 of these genes were regulated in the same direction in sensitive versus resistant cell lines. These 97 genes are listed in Tables 1 and 2.

TABLE 1
Genes down-regulated in Ionafarnib sensitive cell lines
Genes downregulated in sensitive cell lines compared to resistant cell lines

| | | | Fold change |
|---|---|---|---|
| | | | sensitive/resistant |
| row.names | ProbeDescript | Acc.Num | (1 example) |
| 203408_s_at | Homo sapiens special AT-rich sequence binding protein 1 (binds to nuclear matrix/scaffold- | NM_002971 | −1038.654414 |
| | associating DNA quote s) (SATB1), mRNA. | | |
| 204115_at | Homo sapiens guanine nucleotide binding protein (G protein), gamma 11 mRNA, complete | NM_004126 | −231.0241746 |
| | cds. | | |
| 213693_s_at | Mucin-1 | NM_182741 | −17.18645394 |
| 222549_at | Homo sapiens claudin-1 (CLDN1) mRNA, complete cds. | NM_021101 | −15.84438756 |
| 219523_s_at | Homo sapiens mRNA for KIAA1455 protein, partial cds. | NA | −15.65552521 |
| 212560_at | Homo sapiens gp250 precursor, mRNA, complete cds. | NM_003105 | −15.4603385 |
| 238967_at | — | NA | −13.08552588 |
| 203960_s_at | Homo sapiens (clone tec24) mRNA. | NM_016126 | −12.96185235 |
| 207847_s_at | Human polymorphic epithelial mucin (PEM) mRNA, complete cds. | NM_002456 | −12.84023765 |
| 215691_x_at | Homo sapiens (clone tec24) mRNA. | NM_016126 | −11.40030092 |
| 211596_s_at | Homo sapiens mRNA; cDNA DKFZp586O1624 (from clone DKFZp586O1624); partial cds. | NM_015541 | −10.10221677 |
| 216252_x_at | Homo sapiens cDNA FLJ36021 fis, clone TESTI2015568. | NM_000043 | −9.743229174 |
| 202193_at | Homo sapiens LIM domain kinase 2 (LIMK2), transcript variant 2b, mRNA. | NM_005569 | −7.333490312 |
| 209140_x_at | Homo sapiens MHC class I (HLA-B) mRNA, HLA-B*3906 allele, partial cds. | NM_005514 | −7.037192607 |
| 209205_s_at | Homo sapiens LIM domain only 4 (LMO4), mRNA. | NM_006769 | −6.201855873 |
| 218323_at | Homo sapiens cDNA clone IMAGE: 3906539, partial cds. | NM_018307 | −5.320271982 |
| 211715_s_at | Homo sapiens 3-hydroxybutyrate dehydrogenase (heart, mitochondrial), mRNA (cDNA | NM_004051 | −5.032586274 |
| | clone MGC: 2723 IMAGE: 2822178), complete cds. | | |
| 221636_s_at | Homo sapiens mRNA; cDNA DKFZp586G2122 (from clone DKFZp586G2122); complete | NM_017898 | −4.811544258 |
| | cds. | | |
| 219520_s_at | Homo sapiens KIAA1280 protein, mRNA (cDNA clone IMAGE: 5109476). | NM_015691 | −4.629960867 |
| 202551_s_at | Homo sapiens cysteine-rich repeat-containing protein S52 precursor, mRNA, complete | NM_016441 | −4.574455405 |
| | cds. | | |
| 209513_s_at | Homo sapiens chromosome 9 open reading frame 99, mRNA (cDNA clone MGC: 10940 | NM_032303 | −4.507414107 |
| | IMAGE: 3630835), complete cds. | | |
| 212496_s_at | Homo sapiens cDNA FLJ44906 fis, clone BRAMY3007078. | NM_015015 | −4.227193053 |
| 208615_s_at | Homo sapiens cDNA FLJ26067 fis, clone PRS08047.PRL2 | NM_003479 | −4.143931053 |
| 228619_x_at | Human gene from PAC 69E11, chromosome 1. | NM_152902 | −4.015835018 |
| 226148_at | Homo sapiens HSPC063 protein (HSPC063), mRNA. | NM_014155 | −3.944930818 |
| 202777_at | Homo sapiens soc-2 suppressor of clear homolog (C. elegans), mRNA (cDNA clone | NM_007373 | −3.850377772 |
| | MGC: 54207 IMAGE: 6067669), complete cds. | | |
| 211911_x_at | major histocompatibility complex, class I, C /// major histocompatibility complex, class I, C | NM_002117 | −3.839717044 |
| | /// major histocompatibility complex, class I, B /// major histocompatibility complex, class I, B | | |
| 209512_at | Homo sapiens chromosome 9 open reading frame 99, mRNA (cDNA clone MGC: 10940 | NM_032303 | −3.762782763 |
| | IMAGE: 3630835), complete cds. | | |
| 240162_at | Homo sapiens hypothetical gene supported by BC034612 (LOC401169), mRNA. | NA | −3.733684436 |
| 231897_at | Homo sapiens cDNA FLJ34629 fis, clone KIDNE2015515, highly similar to NADP- | NM_012212 | −3.660650402 |
| | DEPENDENT LEUKOTRIENE B4 12-HYDROXYDEHYDROGENASE (LTB4DH)(EC | | |
| | 1.1.1.-). | | |
| 218806_s_at | Homo sapiens VAV-3 protein beta isoform (VAV-3) mRNA, alternatively spliced, complete | MM_006113 | −3.461266436 |
| | cds. | | |
| 1558956_s_at | Homo sapiens KIAA1374 protein, mRNA (cDNA clone IMAGE: 5273080), partial cds. | NM_020800 | −3.407940154 |
| 244881_at | Homo sapiens mRNA for leishmanolysin-like peptidase variant 2 (LMLN gene). | NA | −3.291168752 |
| 233112_at | Homo sapiens cDNA FLJ10263 fis, clone HEMBB1000991. | NM_203403 | −3.233731734 |
| 221218_s_at | Homo sapiens thiamin pyrophosphokinase 1, mRNA (cDNA clone IMAGE: 3622116), partial | NM_022445 | −3.216741478 |
| | cds. | | |
| 229377_at | Growth hormone regulated TBC protein 1 | NM_024719 | −3.183910983 |
| 212495_at | Homo sapiens cDNA FLJ44906 fis, clone BRAMY3007078. | NM_015015 | −3.180161425 |
| 224880_at | Homo sapiens full length insert cDNA clone YZ83E03. | NM_005402 | −3.175535723 |
| 209039_x_at | Homo sapiens clone CDABP0131 mRNA sequence. | NM_006795 | −3.07865456 |
| 224436_s_at | Homo sapiens cDNA FLJ33186 fis, clone ADRGL2004676. | NM_015469 | −3.044699821 |
| 201636_at | Human fragile X mental retardation protein 1 homolog FXR1 mRNA, complete cds. | NA | −2.96493402 |
| 201800_s_at | Homo sapiens oxysterol-binding protein 1 (OSBP1) mRNA, complete cds. | NM_002556 | −2.922088756 |
| 201291_s_at | Homo sapiens cDNA clone IMAGE: 6388518, partial cds. | NM_001067 | −2.820595921 |
| 221543_s_at | Homo sapiens chromosome 8 open reading frame 2, mRNA (cDNA clone | NM_0010033790 | −2.789487332 |
| | IMAGE: 5550432), complete cds. | | |
| 208783_s_at | Homo sapiens cDNA FLJ26049 fis, clone PRS02694. | NM_002389 | −2.779836439 |
| 224641_at | Homo sapiens mRNA for putative 40-2-3 protein. | NM_032288 | −2.690734417 |
| 218926_at | Homo sapiens mRNA; cDNA DKFZp434C0917 (from clone DKFZp434C0917); partial cds. | NM_018657 | −2.615886598 |
| 243141_at | hypothetical protein MGC26963 | NM_152621 | −2.596917173 |
| 225300_at | Homo sapiens cDNA FLJ43777 fis, clone TESTI2051177, highly similar to Homo sapiens | NA | −2.414961183 |
| | TRAF4 associated factor 1. | | |
| 218079_s_at | Homo sapiens mRNA for hypothetical protein, clone JuaW-XI-88. | NM_024835 | −2.279946545 |
| 218671_s_at | Homo sapiens ATPase inhibitory factor 1, transcript variant 1, mRNA (cDNA clone | NM_016311 | −2.236123702 |
| | MGC: 8898 IMAGE: 3877506), complete cds. | | |
| 226154_at | Homo sapiens CGI-04 protein mRNA, complete cds. | NM_005690 | −2.023706403 |
| 211988_at | Homo sapiens cDNA FLJ10670 fis, clone NT2RP2006312, highly similar to Homo sapiens | NM_003079 | −1.941268939 |
| | BAF57 gene. | | |
| 222665_at | Homo sapiens CGI-90 protein, mRNA (cDNA clone MGC: 8729 IMAGE: 3896168), complete | NM_016033 | −1.868266158 |
| | cds. | | |
| 202955_s_at | Homo sapiens brefeldin A-inhibited guanine nucleotide-exchange protein 1 (BIG1), mRNA. | NM_006421 | −1.817178323 |
| 204020_at | H. sapiens mRNA for pur alpha extended 3-primeuntranslated region. | NM_005859 | −1.760518027 |
| 218483_s_at | Homo sapiens mRNA; cDNA DKFZp586N0222 (from clone DKFZp586N0222); complete | NM_020153 | −1.747145791 |
| | cds. | | |
| 215235_at | Homo sapiens full length insert cDNA YO67D02. | NM_003127 | −1.673768498 |
| 223026_s_at | Homo sapiens DC15 (DC15) mRNA, complete cds. | NM_016226 | −1.607701982 |
| 224617_at | ROD1 regulator of differentiation 1 (S. pombe) | NM_005156 | −1.318593615 |
| 224778_s_at | Homo sapiens, clone IMAGE: 5259584, mRNA. | NA | −1.297738767 |
| 218995_s_at | Homo sapiens endothelin 1 (EDN1) | NM_001955 | −3.2 |

TABLE 2
Genes up-regulated in Ionafarnib sensitive cell lines
Genes upregulated in sensitive cell lines v resistant cell lines

| | | | |
|---|---|---|---|
| 204957_at | Homo sapiens origin recognition complex subunit ORC5T (ORC5L) mRNA, alternatively | NM_002553 | 1.714752074 |
| | spliced product, complete cds. | | |
| 224635_s_at | Homo sapiens baculoviral IAP repeat-containing 6 (apolion) (BIRC6), mRNA | NM_016252 | 1.845484985 |
| 224796_at | Homo sapiens mRNA; cDNA DKFZp781K0428 (from clone DKFZp781K0428). | NM_018482 | 1.886876501 |
| 205512_s_at | Homo sapiens programmed cell death 8 (apoptosis-inducing factor) (PDCD8), nuclear | NM_004208 | 2.039195366 |
| | gene encoding mitochondrial protein, transcript variant 2, mRNA. | | |
| 222666_s_at | Homo sapiens cDNA FLJ12842 fis, clone NT2RP2003286, weakly similar to PROBABLE | NM_005772 | 2.041033697 |
| | RNA 3-prime-TERMINAL PHOSPHATE CYCLASE (EC 6.5.1.4). | | |
| 227696_at | Exosome component 6 | NM_058219 | 2.114768877 |
| 225344_at | Homo sapiens mRNA; cDNA DKFZp451D1618 (from clone DKFZp451D1618). | NM_181782 | 2.145223021 |
| 212120_at | Homo sapiens cDNA FLJ25326 fis, clone TST00424. | NM_012249 | 2.381713698 |
| 225070_at | Homo sapiens chromosome 6 open reading frame 68, mRNA (cDNA clone MGC: 70590 | NM_138459 | 2.574156917 |
| | IMAGE: 6500832), complete cds. | | |
| 220890_s_at | Homo sapiens mRNA; cDNA DKFZp564O176 (from clone DKFZp564O176); complete cds. | NM_016355 | 2.804998501 |
| 213315_x_at | Homo sapiens (clone 48A8) mRNA. | NM_178124 | 2.8108374 |
| 218258_at | DNA-DIRECTED RNA POLYMERASE I 16 KDA POLYPEPTIDE. | NM_015972 | 2.828427125 |
| 226255_at | Homo sapiens mRNA; cDNA DKFZp686K0367 (from clone DKFZp686K0367); complete | NM_006777 | 2.867910497 |
| | cds. | | |
| 202371_at | Homo sapiens B lymphocyte activation-related protein mRNA, complete cds. | NM_024863 | 2.975227525 |
| 205077_s_at | Homo sapiens PIG-F mRNA for phosphatidyl-inositol-glycan class F, complete cds. | NM_002643 | 2.983488052 |
| 223095_at | Homo sapiens cDNA: FLJ23440 fis, clone HSI00358. | NM_031484 | 3.043222879 |
| 225583_at | Homo sapiens cDNA FLJ31460 fis, clone NT2NE2001191. | NM_025076 | 3.110399735 |
| 217858_s_at | Homo sapiens clone DNA98593 ALEX3 (UNQ2517) mRNA, complete cds. | NM_016607 | 3.15207093 |
| 219675_s_at | Homo sapiens cDNA FLJ31460 fis, clone NT2NE2001191. | NM_025076 | 3.420482861 |
| 229644_at | — | NA | 3.46606811 |
| 205047_s_at | Homo sapiens cDNA FLJ20372 fis, clone HEP19727, highly similar to M27396 Human | NM_001673 | 3.832537746 |
| | asparagine synthetase mRNA. | | |
| 204981_at | Homo sapiens p45-BWR1A (BWR1-A) mRNA, complete cds. | NM_002555 | 4.272557776 |
| 204793_at | Homo sapiens KIAA0443 mRNA, partial cds. | NM_014710 | 4.952304473 |
| 201811_x_at | Homo sapiens SH3-domain binding protein 5 (BTK-associated) (SH3BP5), mRNA. | NM_004844 | 5.613886903 |
| 201161_s_at | Homo sapiens cold shock domain protein A, mRNA (cDNA clone MGC: 20058 | NM_003651 | 6.752398268 |
| | IMAGE: 4563632), complete cds. | | |
| 205904_at | H. sapiens mRNA for MHC class I mic-B antigen. | NM_000247 | 7.769382824 |
| 213093_at | protein kinase C, alpha | NM_002737 | 8.153955032 |
| 218694_at | Homo sapiens AD032 mRNA, complete cds. | NM_016608 | 9.105258648 |
| 212775_at | Homo sapiens cDNA: FLJ22293 fis, clone HRC04421, highly similar to AF035292 Homo | NA | 10.57070721 |
| | sapiens clone 23584 mRNA sequence. | | |
| 218338_at | Human cation-dependent mannose 6-phosphate-specific receptor mRNA, complete cds. | NM_004426 | 15.41753267 |
| 223087_at | Homo sapiens cDNA FLJ40827 fis, clone TRACH2011500. | NM_018479 | 44.09979402 |
| 224461_s_at | Homo sapiens mRNA; cDNA DKFZp686H14188 (from clone DKFZp686H14188). | NM_032797 | 49.97033664 |
| 209189_at | Homo sapiens mRNA; cDNA DKFZp686J04124 (from clone DKFZp686J04124). | NM_005252 | 52.65505058 |
| 213417_at | Homo sapiens cDNA FLJ10169 fis, clone HEMBA1003662, highly similar to TBX2 | NM_005994 | 62.90924041 |
| | PROTEIN. | | |
| 205226_at | Homo sapiens AT2 receptor-interacting protein 1 mRNA, complete cds. [NetAFFX | NM_006207 | 99.47071146 |
| | PDGFRL) | | |
| 207156_at | Homo sapiens O-acyltransferase (membrane bound) domain containing 1 (OACT1), | NM_021064 | 105.5951443 |
| | mRNA. | | |
| 210664_s_at | Homo sapiens tissue factor pathway inhibitor (lipoprotein-associated coagulation inhibitor). | NM_006287 | 619.0448327 |
| | mRNA (cDNA clone MGC: 9251 IMAGE: 3902987), complete cds. | | |

“Acc. Num.” indicates the public accession number for the indicated biomarker.
Column titles are for each table are identical to that indicated for Table 1.

### Example 2

### Lonafarnib Sensitivity Correlates with Biomarker Expression

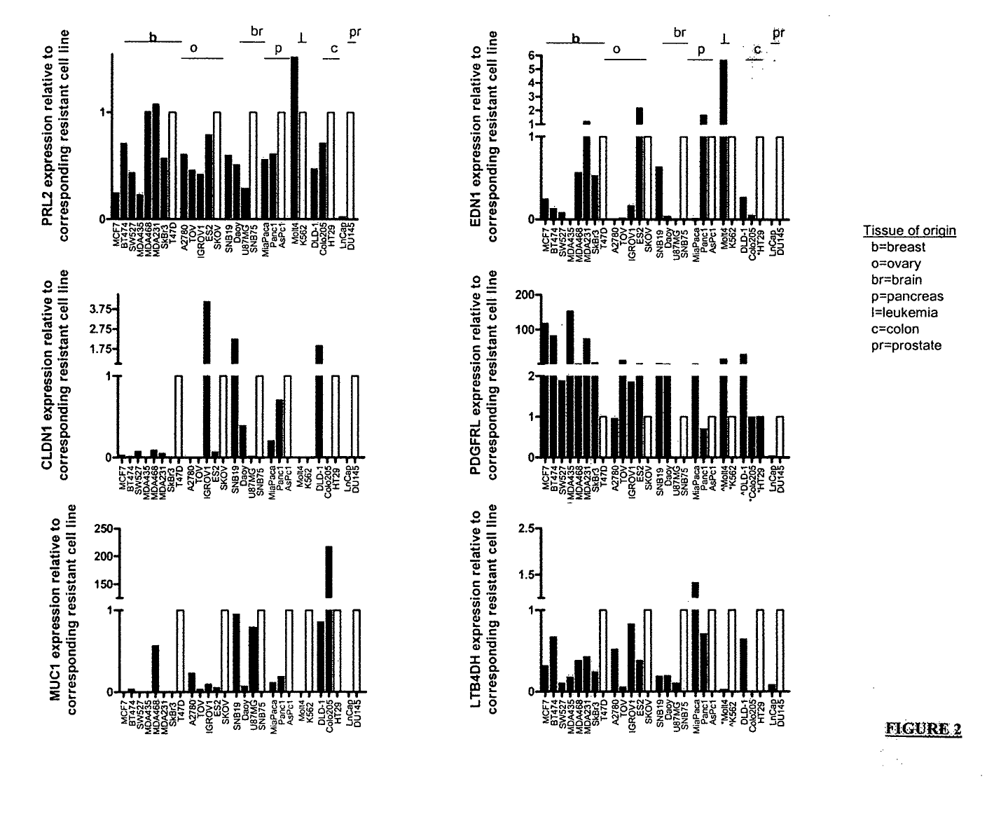

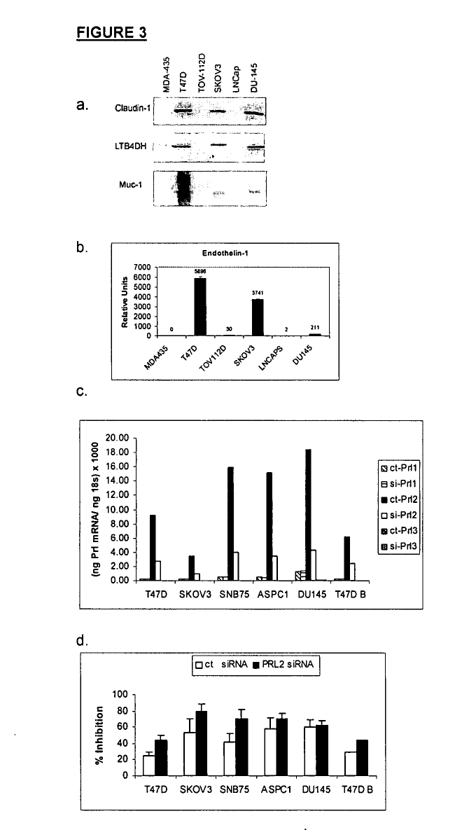

Analysis of the microarray data resulted in a gene list of 98 genes that were differential regulated in sensitive vs. resistant cell lines, n both the breast and the ovarian derived samples. Twenty two of these genes were chosen for follow up, based on a combination of statistical significance, robust expression and biological interests. The 22 genes which were the subject of the follow up investigation were: PRL2, claudin-1, LIM kinase 2, NM_{—}211596, ZTNF2, FRAG1, Mucin-1, NM_{—}224461, PDGFRL, TBX2, PDCD8, ARMCX1, APLP2, XRN1, HLAC, CRIM, LTB4DH, SLC3A2, NLGN4AX, affimextix id. 242346-X-AT (AK124454), TIGA, OPN3, ODAG, RBMX2, MOSPD1 and ARD1A. Gene expression for these 22 genes was confirmed by RT-PCR in the 5 cell lines and then expanded to a larger panel of 22 additional cell lines (FIG. 1). The expression pattern of six genes was found to be consistently differentially regulated (when comparing sensitive to resistant cell lines) in at least 80% of the expanded panel. Sensitive cell lines had relatively low gene expression of PRL2, claudin-1, mucin-1, LTB4DH and endothelin-1 and relatively high gene expression of PDGFRL. FIG. 2 sets forth the level of expression of each of the 6 selected genes in 22 cell lines. Where antibodies were available, the expression pattern of each encoded protein was evaluated. Celts that were relatively resistant to lonafarnib had elevated levels of claudin-1, mucin-1, LTB4DH and endothelin-1. FIGS. 3 (a) and (b) set forth the protein expression level observed for each. FIG. 3 (a) is a Western blot wherein the level of protein expression for claudin-1, LTB4DH and mucin-1 was determined in six cell lines. FIG. 3(b) is ELISA data wherein the level of endothelin-1 secreted from a host cell was determined in six cell lines.

Though PRL2 has been observed to be expressed at relatively high levels in resistant cells, depletion of PRL2 mRNA, in cells, was observed, in turn, to increase the level of lonafarnib sensitivity. PRL2 mRNA expression was depleted using PRL2 siRNA. The data generated in this work is set forth in FIGS. 3(c) and (d). FIG. 3 (c) sets forth the level of PRL1, PRL2 and PRL2 mRNA expression observed in six different cell lines exposed to PRL2 siRNA. The level of PRL1 and PRL3 expression was unaffected by exposure to PRL2 siRNA, whereas the level of PRL2 was reduced. FIG. 3 (d) sets forth the level of lonafarnib sensitivity in cells exposed to PRL2 siRNA or to a control siRNA. The level of growth inhibition was observed to increase when PRL2 mRNA levels were depleted by exposure to PRL2 siRNA.

Endothelian-1 ELISA. Media from cells was collected and analyzed by QuantiGlo ELISA (R&D Systems, Minneapolis, Minn.). 100 μl of media sample was mixed with 100 μl buffer and the mixture was added to a microplate coated with immobilized anti-ET1 antibody. The microplate was incubated at room temperature for 1.5 hours while shaking at 500 rpm. Samples in the microplate were then washed four times with 400 μl wash buffer, 200 μl ET-1 conjugate, comprising anti-ET-1 antibody complexed with horseradish peroxidase, was added to the samples and they were incubated at room temperature for 3 hours while shaking at 500 rpm. The samples were then washed four times with 400 μl wash buffer. 100 μl Glo reagent was added to the samples. Luminescence was measured and relative levels were graphed.

Cell Culture. The human cancer cell lines MCF-7, MDA-MB468 (MDA-468), MDA-MB-231 (MDA-231), SKBr-3, BT-474, T47D, SW527, ES2, SKOV-3, TOV-112D, IGROV-1, LNCap, DU145, SNB19, SNB75, Daoy, U87MG, MiaPaca, PANC1, AsPc1, K562, Molt4, DLD-1, Colo-205, HT29 (American Type Culture Collection, Manassas, Va.), MDA-MB-435 (MDA-435) and A2780 (National Cancer Institute, Bethesda, Md.) were maintained in 1:1 mixture of DME:F12 supplemented with 2 mM glutamine, 50 units/ml penicillin, 50 units/ml streptomycin, and 10% heat inactivated fetal bovine serum (Invitrogen, Carlsbad, Calif.) and incubated at 37° C. in 5% CO₂.

Growth assays. Soft agar assays were performed in 6-well dishes by seeding 10,000-20,000 cells in each well. Cells were plated in top 0.35% low melting point agarose in DMEM with 10% fetal bovine serum over a bottom 0.6% agarose feeding layer. Cells were grown in the presence of lonafarnib for 14 days and colonies were stained with 1 mg/ml MTT (dimethylthiazol-diphenyl-terrazolium bromide) in PBS. The plates were scanned and the colony area was determined as the sum of the areas stained by MTT.

Protein Analysis. Cells were lysed in RIPA buffer (50 mM Tris-HCl, 50 mM NaCl, 1% NP40, 0.5% Na-deoxycholate, 1 mM EDTA, 2.5 mM Na₃VO₄, 20 mM beta-glycerol phosphates and complete protease inhibitor (Roche, Indianapolis, Ind.)) and cleared by centrifugation. Protein concentration was determined using BCA reagent (Pierce Chemical Co., Rockford, Ill.). Samples were separated by 8, 10, or 14% SDS-PAGE (Invitrogen), transferred to polyvinylidene difluoride (PVDF) membrane, immunoblotted and detected by chemiluminescence using ECL detection reagents (Amersham, Piscataway, N.J.). Polyclonal antibodies used. Akt P-Akt (Ser-473), MARK, mucin- and P-MAPK (Thr202/Tyr204) (Cell Signaling, Beverly, Mass.), claudin-1 (Invitrogen; Carlsbad, Calif.), and LTB4DH (Abnova, Taipei City, Taiwan), Monoclonal antibody used: HDJ-2 (human DnaJ) (Neomarkers, Fremont, Calif.).

FPT Assay. Protein cell lysates were incubated with 225 nM [³H] FPP [16.1 ci/mmol] (Perkin Elmer Life Sciences, Wellesley, Mass.) in assay buffer (50 mM Tris, 5 mM MgCl₂, 5 μM ZnCl₂, 0.1% Triton-X 100, 5 mM dithiolthreitol) along with 100 nM biotinylated peptide substrate (DESGPGCMSCKCVLS) (SEQ ID NO: 16) (synthesized by Syn-Pep, Dublin, Calif.). After 1 hour, the reaction was stopped with 750 μg streptavidin-coated beads (Amersham) in 0.25M EDTA and product ([³H] prenyl peptide) formation was measured using scintillation proximity assay.

PRL2 siRNA Transfection. Cells were transiently transfected overnight with 100 nM siRNA and 50 ul Lipofectamine 2000 (Invitrogen). Dharmacon (Chicago, Ill.) siRNAs were used: control siRNA#1, PRL2 (GAAAUACCGACCUAAGAUGUU (SEQ ID NO: 17), and 5′-p-CAUCUUAGGUCGGUAUUUCUU (SEQ ID NO: 18)), and PRL2b (CGACUUUGGUUCGAGUUUGUU (SEQ ID NO: 19) and 5′-p-CAAACUCGAACCAAAGUCGUU (SEQ ID NO: 20)). Cells were trypsinized and plated at 4,000-8,000 cells per well in a 6-well plate. 6 days later cells were stained with crystal violet (Sigma-Aldrich; St. Louis, Mo.). The plates were scanned and the colony area was determined as the sum of the areas stained by crystal violet.

Quantitative PCR. Quantitative, real-time PCR was performed on an AB17900 machine (Applied Biosystems, Foster City, Calif.), using the BIO-RAD iScript Custom one-step RT-PCR Kit for Probes with ROX. (Hercules, Calif.). Primer and probe were designed using ABI Primer Express 2.0, except EDN1 which was designed using the Universal Probe Library Assay Design Center (Roche Applied Sciences, Basel, Switzerland). The probes and primers used for the six genes in FIG. 1 are as follows:

| | |
|---|---|
| LTB4DH Forward Primer: | |
| 121/CACTGTTATCGGCCAGATGAAG; | (SEQ ID NO: 21) |
| Reverse Primer: | |
| 198/GGGCCGGTTCTGTTATATGTAGA; | (SEQ ID NO: 22) |
| Probe (FAM-TAMRA): | |
| 151/AAGGATTGCCATATGTGGAGCCAT; | (SEQ ID NO: 23) |
| Endothelin.1 Forward Primer: | |
| 367/GCTCGTCCCTGATGGATAAA; | (SEQ ID NO: 24) |
| Reverse Primer: | |
| 436/CCATACGGAACAACGTGCT; | (SEQ ID NO: 25) |

Probe (FAM-NFQ) from Roche Universal Probe
Library (Roche Diagnostics, Base,
Switzerland) #29:

| | |
|---|---|
| CTTCTGCC; | (SEQ ID NO: 26) |
| PRL2 Forward primer: | |
| GTCCAGGCAGTGAGCGTACTT; | (SEQ ID NO: 27) |
| Reverse primer: | |
| AATTTTAAGCTACCAGCATTCTCTCA; | (SEQ ID NO: 28) |
| Probe (FAM-TAMRA): | |
| CGTTACTCTGATTTTCTGTCTAG; | (SEQ ID NO: 29) |
| Mucin-1 Forward primer: | |
| CTGCTGGTGCTGGTCTGTGT; | (SEQ ID NO: 30) |
| Reverse primer: | |
| ATGTCCAGCTGCCCGTAGTT; | (SEQ ID NO: 31) |
| Probe (FAM-AMRA): | |
| CATTGCCTTGGCTGTC; | (SEQ ID NO: 32) |
| Claudin-1 Forward primer: | |
| AATCCAACAGCAAGGGAGATTTT; | (SEQ ID NO: 33) |
| Reverse primer: | |
| AGCGTCAGCTGCCAGCTAAC; | (SEQ ID NO: 34) |
| Probe (FAM-TAMRA): | |
| TCATAAGGTGCTATCTGTTCA; | (SEQ ID NO: 35) |
| PDGFRL Forward primer: | |
| CCGATGTGGAGGTGGAGTTC; | (SEQ ID NO: 36) |
| Reverse primer: | |
| TCCCAGTCCTCTGTGGATCA; | (SEQ ID NO: 37) |
| Probe (FAM-TAMRA): | |
| CCTGTGACGATCCAAGA; | (SEQ ID NO: 38) |

The present invention is not to be limited in scope by the specific embodiments described herein. Indeed, various modifications of the invention in addition to those described herein will become apparent to those skilled in the art from the foregoing description. Such modifications are intended to fail within the scope of the appended claims.

Patents, patent applications, publications, product descriptions, and protocols are cited throughout this application, the disclosures of which are incorporated herein by reference in their entireties for all purposes.

We claim:

1. A method for treating cancer, in a patient, comprising:
(a) determining if a cell mediating said cancer is sensitive to a farnesyl protein transferase inhibitor, wherein the cell is determined to be sensitive to the inhibitor if at least one biomarker selected from those set forth in Table 1, PRL2, claudin-1, mucin-1, LT84DH and endothelin-1 is underexpressed by the cell; and/or at least one biomarker selected from those set forth in Table 2 and PDGFRL is overexpressed by the cell, relative to expression of the biomarker by a farnesyl protein transferase inhibitor resistant cell; and
(b) administering, to said patient, a therapeutically effective amount of a farnesyl protein transferase inhibitor if the cell is sensitive.

2. The method of claim 1 wherein the cell mediates a cancer which is a member selected from the group consisting of lung cancer, lung adenocarcinoma, non small cell lung cancer, pancreatic cancer, exocrine pancreatic carcinoma, colon cancer, colorectal carcinoma, colon adenocarcinoma, colon adenoma, myeloid leukemia, acute myelogenous leukemia (AML), chronic myelogenous leukemia (CML), and chronic myelomonocytic leukemias (CMML), thyroid follicular cancer, myelodysplastic syndrome (MDS), bladder carcinoma, epidermal carcinoma, melanoma, breast cancer, prostate cancer, head and neck cancer, squamous cell cancer of the head and neck, ovarian cancer, brain cancer, glioma, cancers of mesenchymal origin, fibrosarcomas, rhabdomyosarcomas, sarcomas, tetracarcinomas, neuroblastomas, kidney carcinomas, hepatomas, non-Hodgkin's lymphoma, multiple myeloma, and anaplastic thyroid carcinoma.

3. The method of claim 1 wherein the farnesyl protein transferase inhibitor is one or more members selected from the group consisting of:

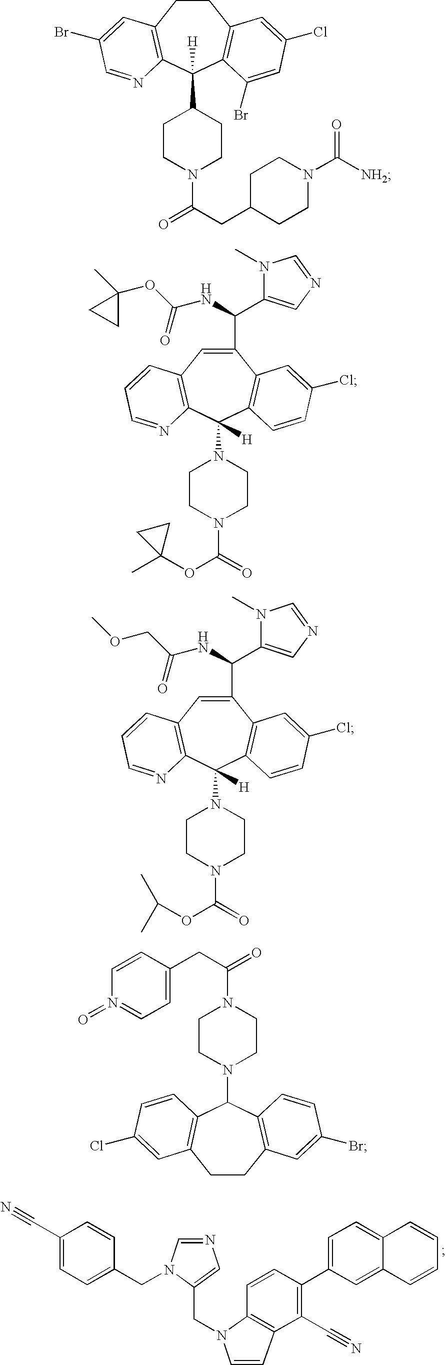

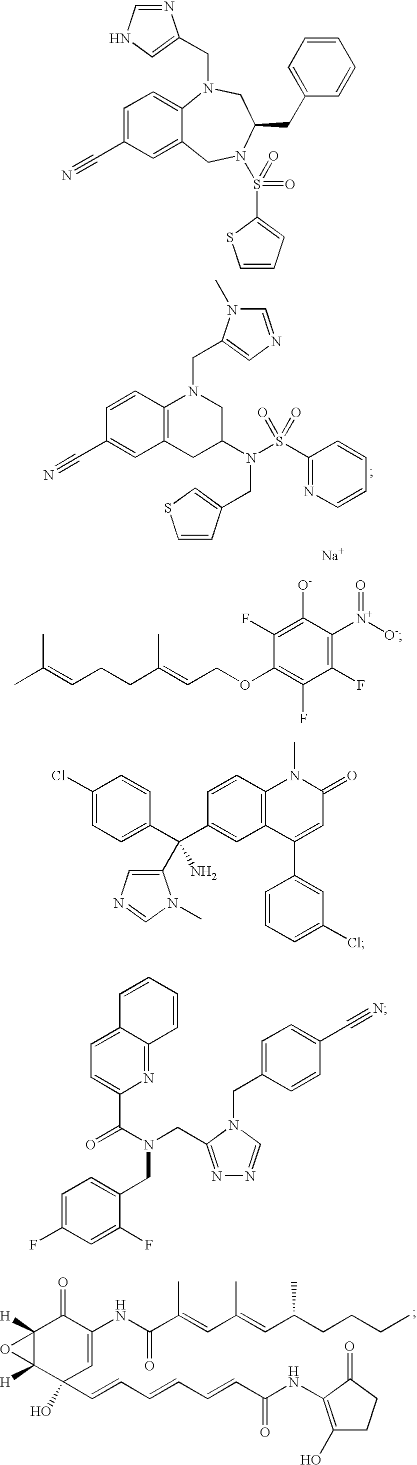

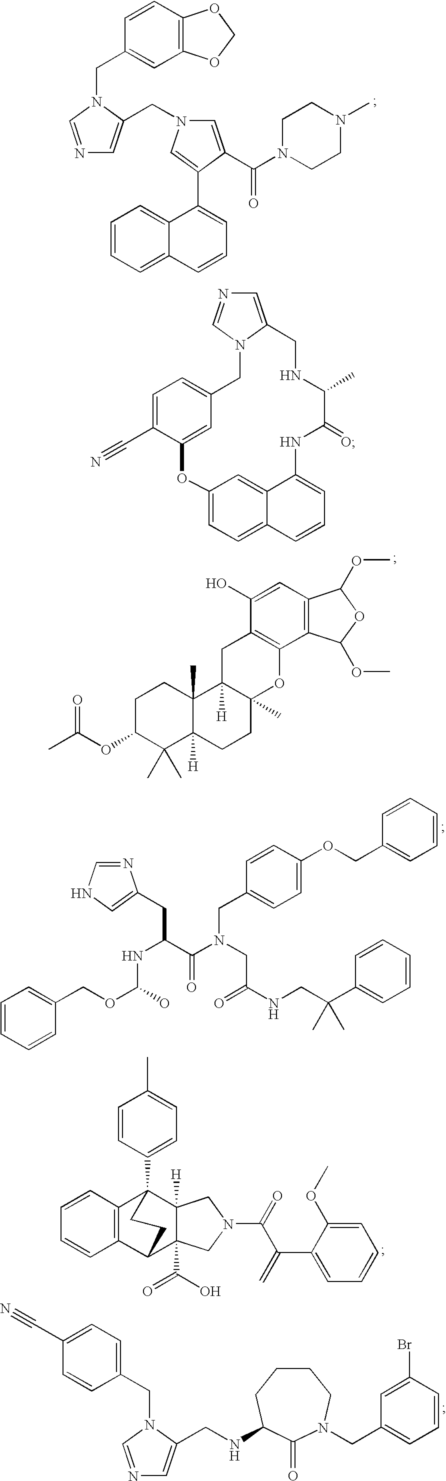

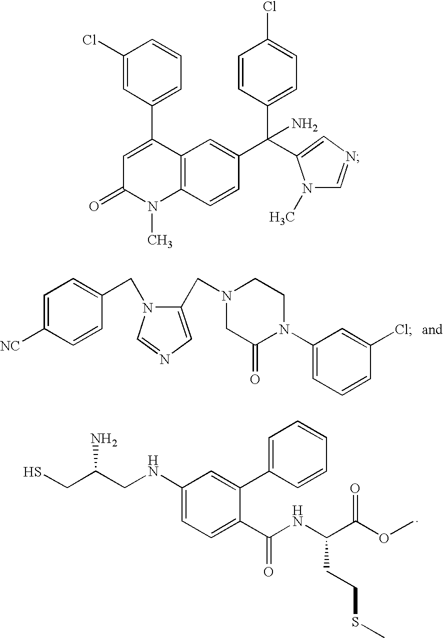

4. The method of claim 1 wherein the patient is administered the farnesyl protein transferase inhibitor in association with a further chemotherapeutic agent or a therapeutic procedure.

5. The method of claim 4 wherein the further chemotherapeutic agent is one or more members selected from the group consisting of paclitaxel, imatinib, gemcitabine, trastuzumab, cisplatin, docetaxel, doxorubicin, melphalan and 5-fluorouracil.

6. A method for assessing whether a farnesyl protein transferase inhibitor inhibits in vitro or in vivo growth or survival of a neoplastic cell comprising determining if said cell underexpresses at least one biomarker selected from the group consisting of PRL2, claudin-1, mucin-1, LTB4DH or endothelin-1 and those set forth in Table 1 and/or overexpresses at least one biomarker selected from the group consisting of PDGFRL and those set forth in Table 2, relative to expression of said biomarker in a cell resistant to said farnesyl protein transferase inhibitor; wherein the inhibitor is determined to inhibit said growth or survival if said underexpression or overexpression is observed.

7. The method of claim 6 wherein said cell is obtained from an animal patient and wherein, the patient is administered a therapeutically effective amount of the farnesyl protein transferase inhibitor if said inhibitor is determined to inhibit said growth or survival.

8. The method of claim 7 wherein said farnesyl protein transferase inhibitor is administered in association with a further chemotherapeutic agent or a therapeutic procedure.

9. The method of claim 6 wherein the neoplastic cell mediates a medical condition selected from the group consisting of lung cancer, lung adenocarcinoma, non small cell lung cancer, pancreatic cancer, exocrine pancreatic carcinoma, colon cancer, colorectal carcinoma, colon adenocarcinoma, colon adenoma, myeloid leukemia, acute myelogenous leukemia (AML), chronic myelogenous leukemia (CML), and chronic myelomonocytic leukemias (CMML), thyroid follicular cancer, myelodysplastic syndrome (MDS), bladder carcinoma, epidermal carcinoma, melanoma, breast cancer, prostate cancer head and neck cancer, squamous cell cancer of the head and neck, ovarian cancer, brain cancer, glioma, cancers of mesenchymal origin, fibrosarcomas, rhabdomyosarcomas, sarcomas, tetracarcinomas, neuroblastomas, kidney carcinomas, hepatomas, non-Hodgkin's lymphoma, multiple myeloma, and anaplastic thyroid carcinoma.

10. The method of claim 6 wherein the farnesyl protein transferase inhibitor is one or more members selected from the group consisting of:

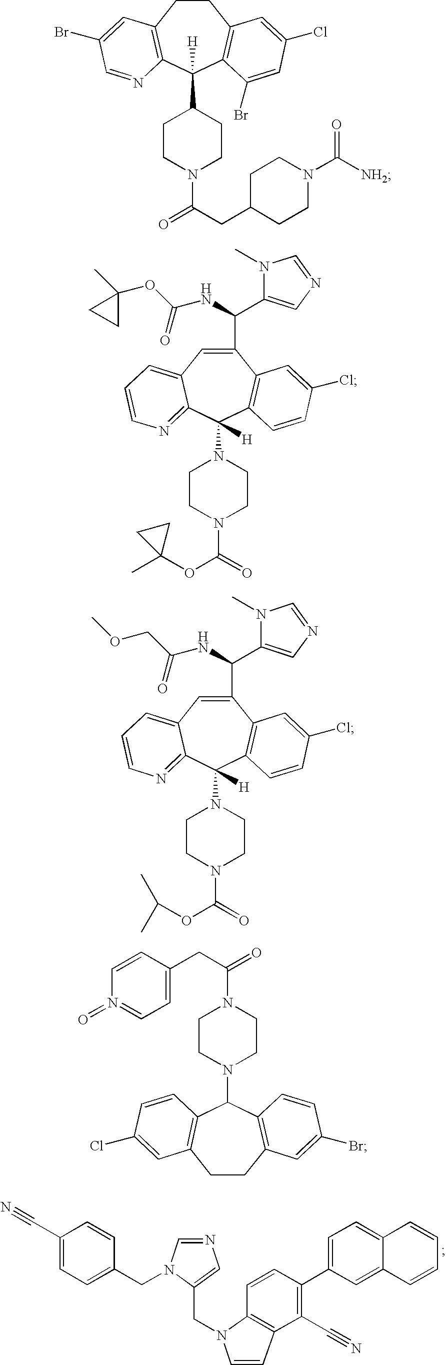

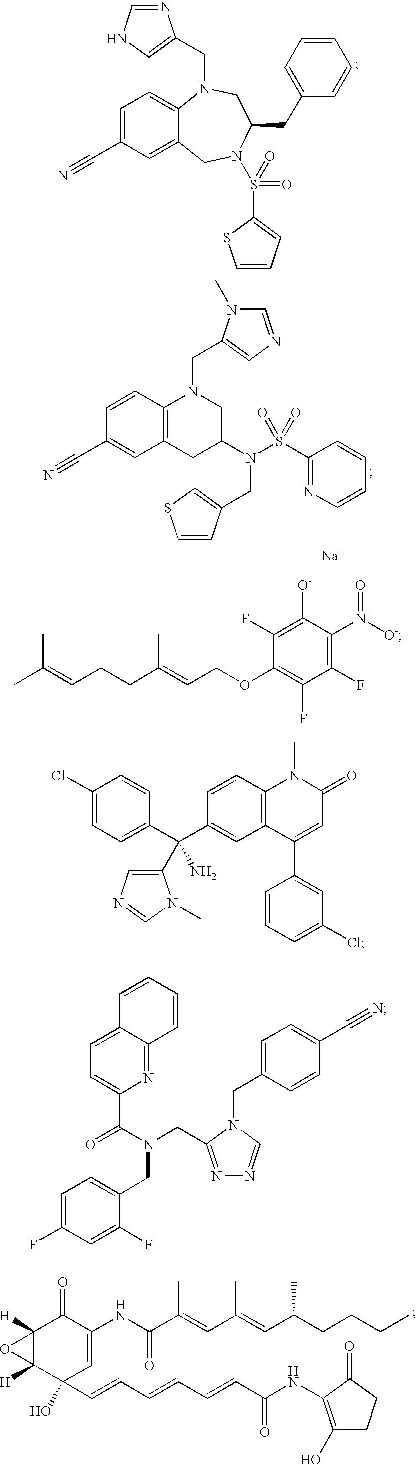

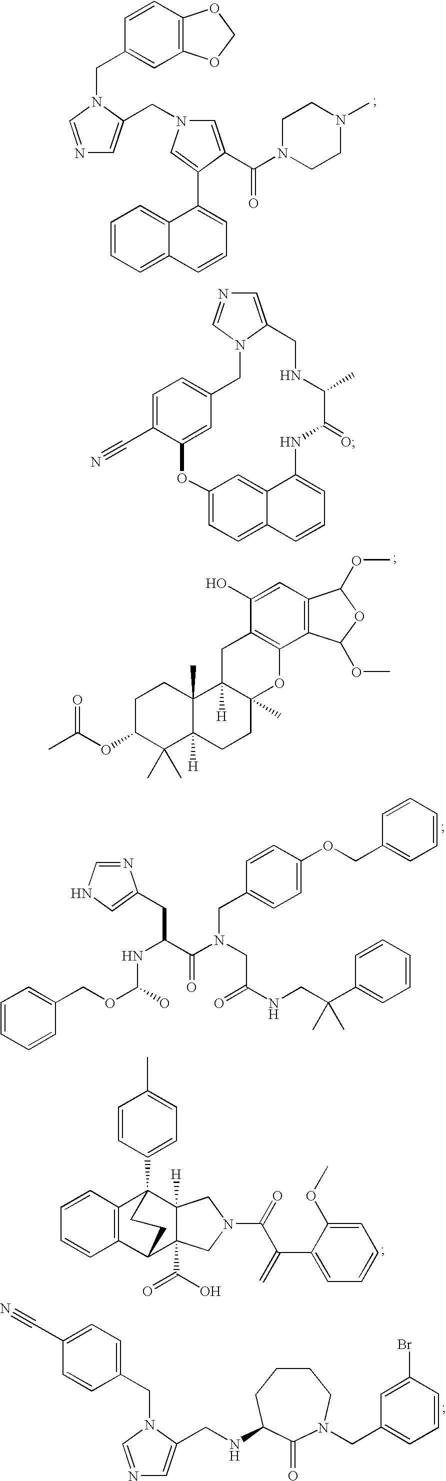

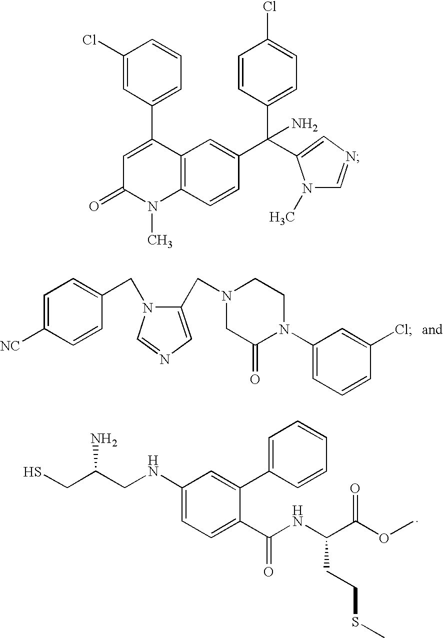

11. A method for selecting a patient with a cancerous condition responsive to a farnesyl protein transferase inhibitor for treatment with said inhibitor comprising determining if a cell which mediates said condition and which is obtained from said patient underexpresses at least one biomarker selected from the group consisting of PRL2, claudin-1, mucin-1, LT84DH, endothelin-1 and those set forth in Table 1 and/or overexpresses at least one biomarker selected from the group consisting of PDGFRL and those set forth in Table 2, relative to farnesyl protein transferase resistant cell expression of the biomarker; wherein the patient is selected if said underexpression or overexpression is observed.

12. The method of claim 11 wherein the patient is administered a therapeutically effective amount of the farnesyl protein transferase inhibitor if said patient is selected.

13. The method of claim 12 wherein said farnesyl protein transferase inhibitor is administered in association with a further chemotherapeutic agent or a therapeutic procedure.

14. The method of claim 11 wherein the cancerous condition is selected from the group consisting of a cancer, lung adenocarcinoma, non small cell lung cancer pancreatic cancer, exocrine pancreatic carcinoma, colon cancer, colorectal carcinoma, colon adenocarcinoma, colon adenoma, myeloid leukemia, acute myelogenous leukemia (AML) chronic myelogenous leukemia (CML), and chronic myelomonocytic leukemias (CMML), thyroid follicular cancer, myelodysplastic syndrome (MDS), bladder carcinoma, epidermal carcinoma, melanoma, breast cancer, prostate cancer, head and neck cancer, squamous cell cancer of the head and neck, ovarian cancer, brain cancer, glioma, cancers of mesenchymal origin, fibrosarcomas, rhabdomyosarcomas, sarcomas, tetracarcinomas, neuroblastomas, kidney carcinomas, hepatomas, non-Hodgkin's lymphoma, multiple myeloma, and anaplastic thyroid carcinoma.

15. The method of claim 11 wherein the farnesyl protein transferase inhibitor is one or more members selected from the group consisting of:

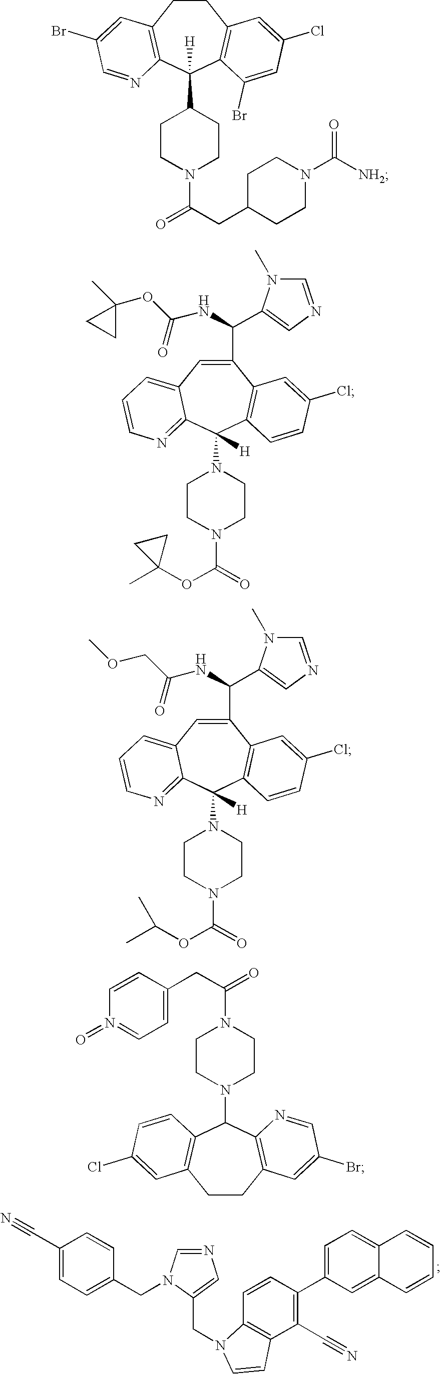

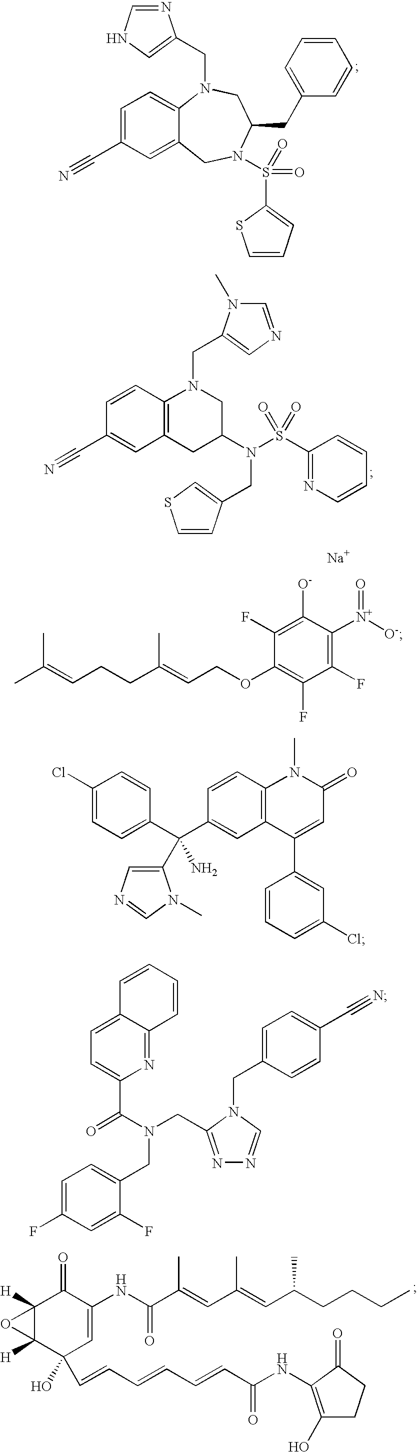

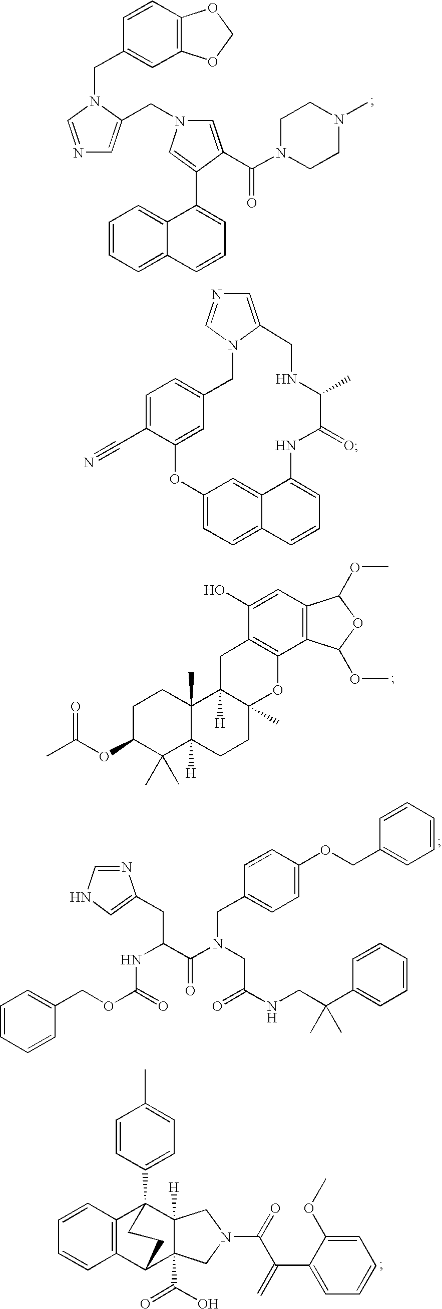

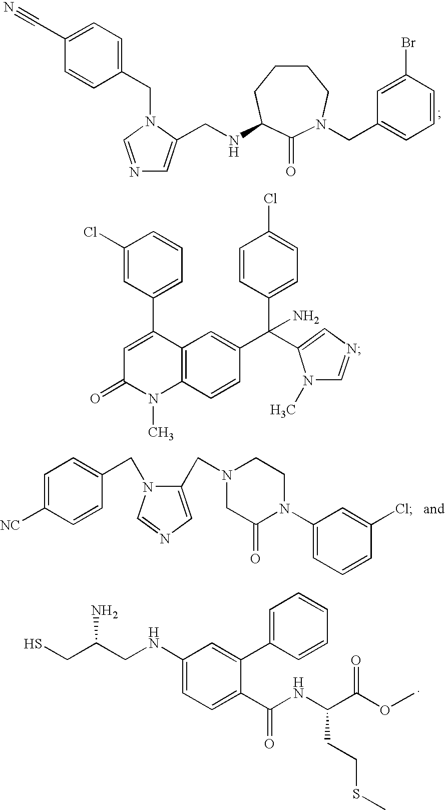

16. A method for selecting a farnesyl protein transferase inhibitor therapy to treat a cancerous condition in a patient comprising determining if a cell taken from said patient which mediates said cancerous condition underexpresses at least one biomarker selected from the group consisting of PRL2, claudin-1, mucin-1, LTB4DH, endothelin-1 and those set forth in Table 1 and/or overexpresses at least one biomarker selected from the group consisting of PDGFRL and those set forth in Table 2, relative to expression of the biomarker in a cell resistant to said inhibitor; wherein the inhibitor is selected for the therapy if said underexpression or overexpression is observed.

17. The method of claim 16 wherein the patient is administered a therapeutically effective amount of the farnesyl protein transferase inhibitor if said inhibitor is selected for the therapy.

18. The method of claim 17 wherein said farnesyl protein transferase inhibitor is administered in association with a further chemotherapeutic agent or a therapeutic procedure.

19. The method of claim 16 wherein the cancerous condition is a member selected from the group consisting of lung cancer, lung adenocarcinoma, non small cell lung cancer, pancreatic cancer, exocrine pancreatic carcinoma, colon cancer, colorectal carcinoma, colon adenocarcinoma, colon adenoma, myeloid leukemia, acute myelogenous leukemia (AML), chronic myelogenous leukemia (CML), and chronic myelomonocytic leukemias (CMML), thyroid follicular cancer, myelodysplastic syndrome (MDS), bladder carcinoma, epidermal carcinoma, melanoma, breast cancer, prostate cancer, head and neck cancer, squamous cell cancer of the head and neck, ovarian cancer, brain cancer, glioma, cancers of mesenchymal origin, fibrosarcomas, rhabdomyosarcomas, sarcomas, tetracarcinomas, neuroblastomas, kidney carcinomas, hepatomas, non-Hodgkin's lymphoma, multiple myeloma, and anaplastic thyroid carcinoma.

20. The method of claim 16 wherein the farnesyl protein transferase inhibitor is one or more members selected from the group consisting of:

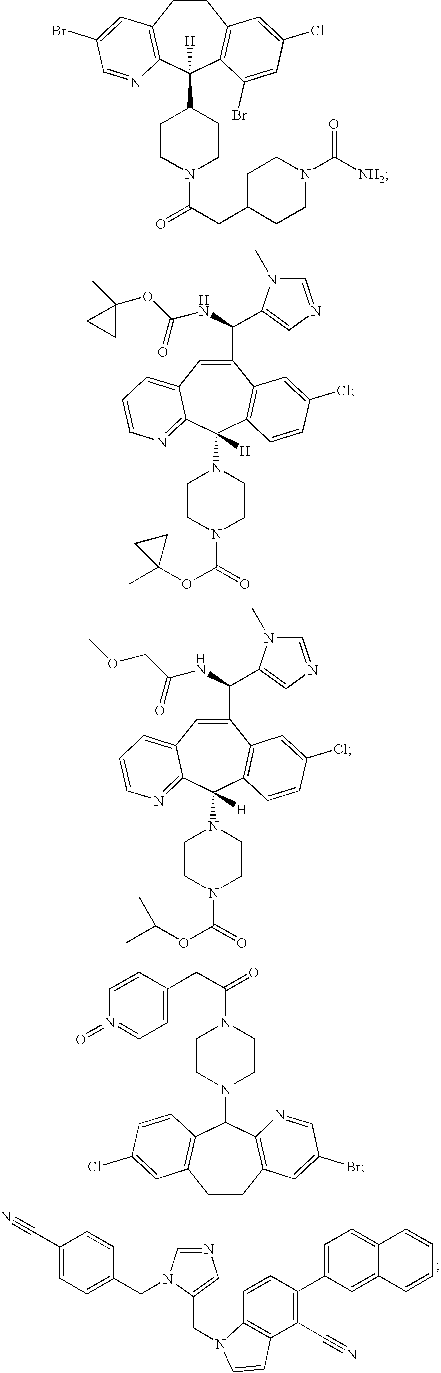

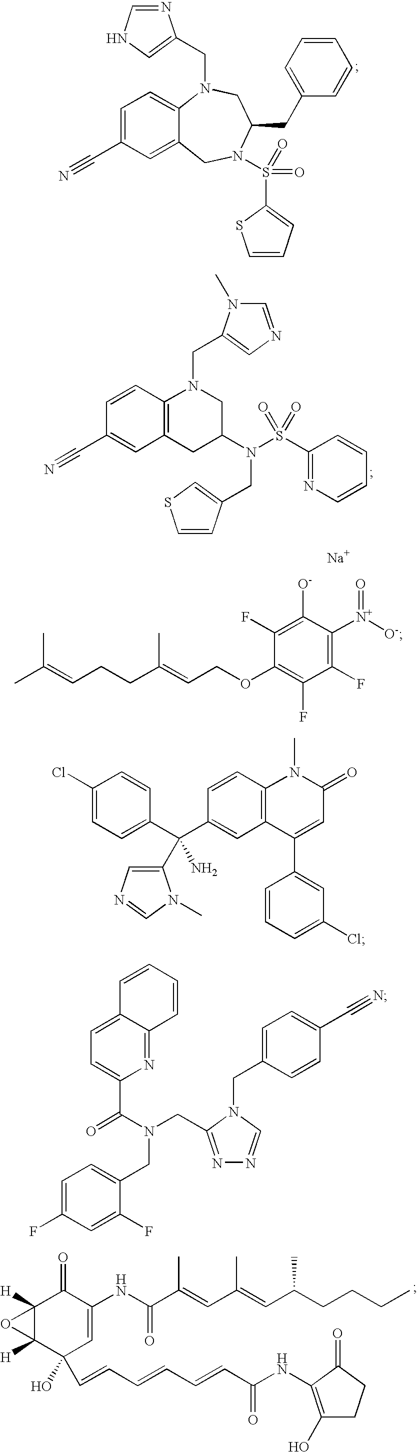

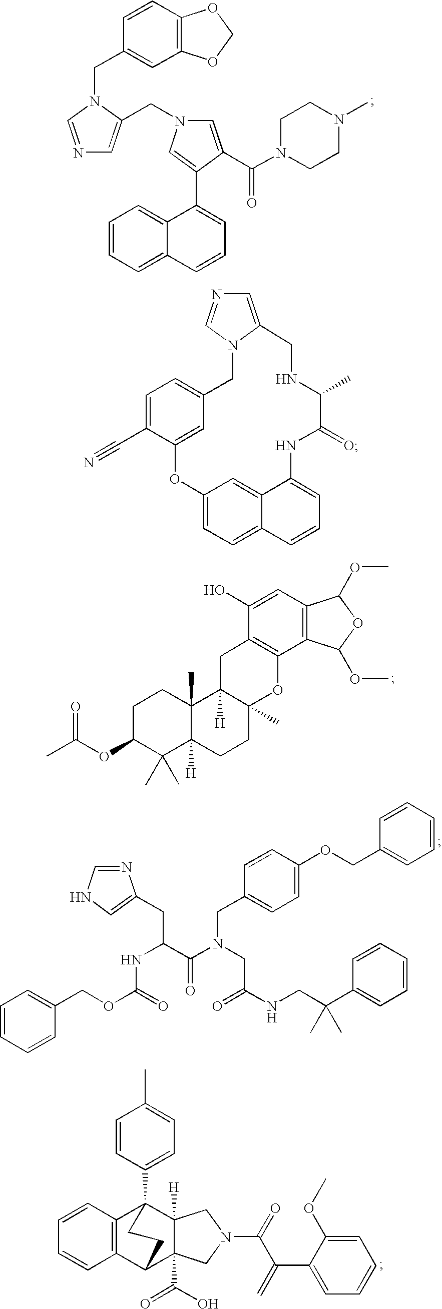

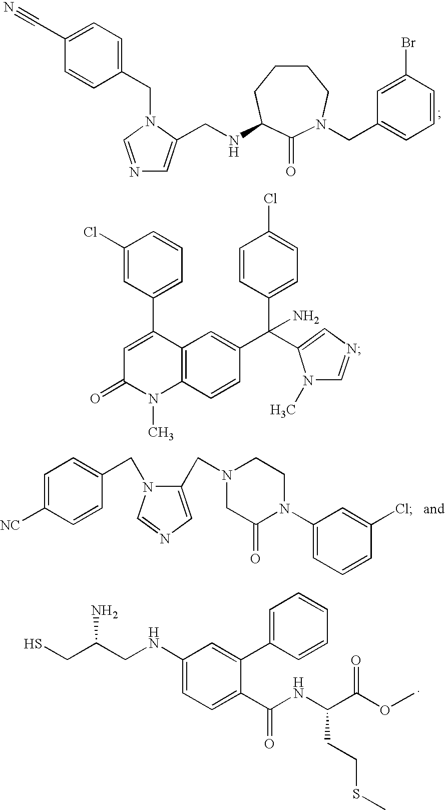

21. A method for diagnosing whether a patient has a cancerous condition that will respond to therapy with a farnesyl protein transferase inhibitor comprising determining, in a cell taken from said patient which mediates said cancerous condition, a level of expression of at least one biomarker selected from the group consisting of any set forth in Table 1, any set forth in Table 2, PDGFRL, PRL2, claudin-1, mucin-1, LTB4DH and endothelin-1; wherein, if any set forth in Table 1, PRL2, claudin-1, mucin-1, LTB4DH or endothelin-1 is underexpressed; and/or if any set forth in Table 2 or PDGFRL is overexpressed, relative to a cell that is resistant to the inhibitor, the condition in the patient is diagnosed as responsive to the inhibitor.

22. The method of claim 21 wherein the patient is administered a therapeutically effective amount of the farnesyl protein transferase inhibitor if said patient is diagnosed as responsive to the inhibitor.

23. The method of claim 22 wherein said farnesyl protein transferase inhibitor is administered in association with a further chemotherapeutic agent or a therapeutic procedure.

24. The method of claim 21 wherein the cancerous condition is a member selected from the group consisting of lung cancer, lung adenocarcinoma, non small cell lung cancer, pancreatic cancer, exocrine pancreatic carcinoma, colon cancer, colorectal carcinoma, colon adenocarcinoma, colon adenoma, myeloid leukemia, acute myelogenous leukemia (AML), chronic myelogenous leukemia (CML), and chronic myelomonocytic leukemias (CMML), thyroid follicular cancer, myelodysplastic syndrome (MDS), bladder carcinoma, epidermal carcinoma, melanoma, breast cancer, prostate cancer, head and neck cancer, squamous cell cancer of the head and neck, ovarian cancer, brain cancer, glioma, cancers of mesenchymal origin, fibrosarcomas, rhabdomyosarcomas, sarcomas, tetracarcinomas, neuroblastomas, kidney carcinomas, hepatomas, non-Hodgkin's lymphoma, multiple myeloma, and anaplastic thyroid carcinoma.

25. The method of claim 21 wherein the farnesyl protein transferase inhibitor is one or more members selected from the group consisting of: 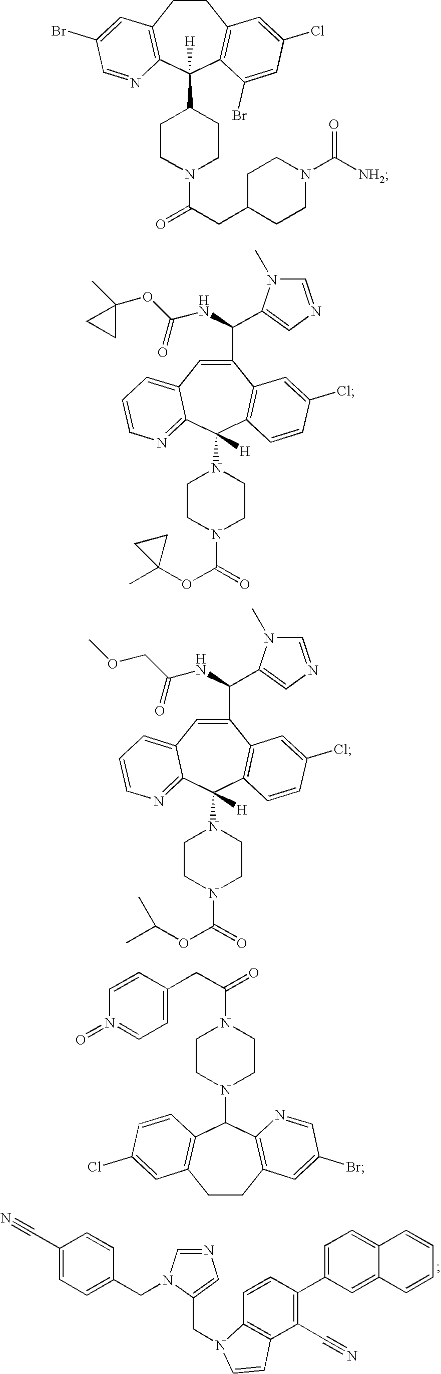 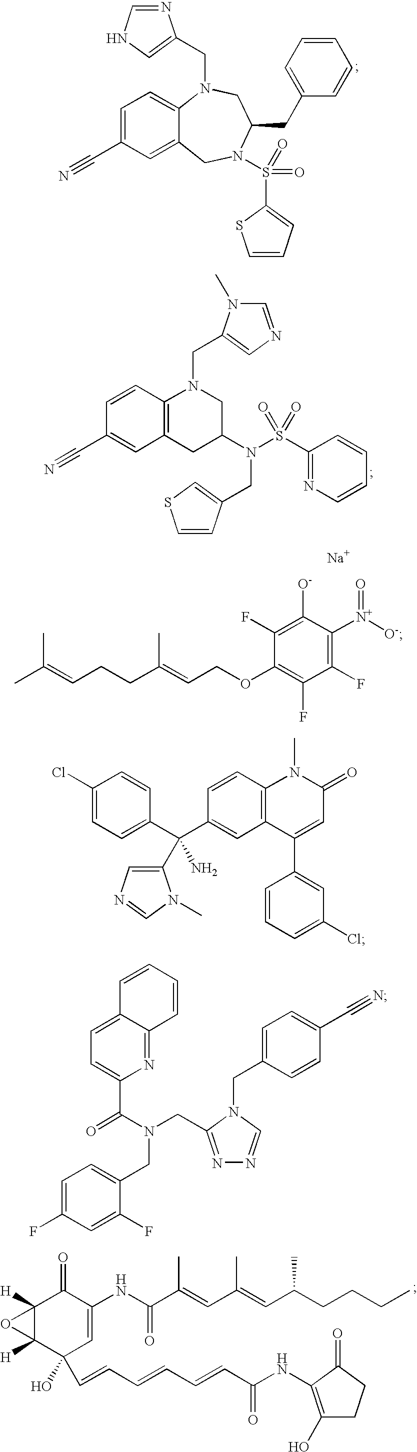 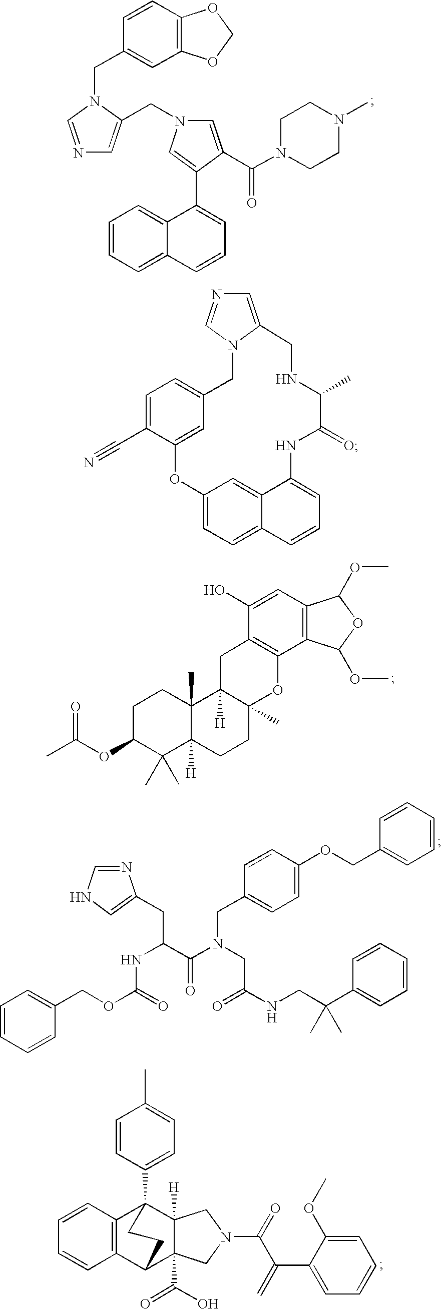 [chemical structure]
